(19) United States
(12) Patent Application Publication
(10) Pub. No.: US 2012/0184977 A1
(43) Pub. Date: Jul. 19, 2012

(54) Device and Method for Crossing Occlusions

(75) Inventor: Yehuda G. Wolf, Mevaseret Zion (IL)

(73) Assignee: SAFEBACK RE-ENTRY MEDICAL LTD., Migdal Haemek (IL)

(21) Appl. No.: 13/156,605

(22) Filed: Jun. 9, 2011

Related U.S. Application Data
(63) Continuation-in-part of application No. 12/993,326, filed on Jan. 18, 2011.

(51) Int. Cl. A61B 17/22

(52) U.S. Cl. 606/159

(57) ABSTRACT

A catheter for crossing occlusions in vessels of a patient. The catheter has a main longitudinal axis, and includes: at least one open-bore type lumen with a distal end and a proximal end, interconnected by a main longitudinal axis, substantially parallel to the main longitudinal axis of the catheter; at least one guide-wire, accommodated within the lumen, reciprocally maneuvered along and around the main longitudinal axis of the lumen; at least one effecter having a distal end and a proximal end, the effecter having at least one active configuration and at least one inactive configuration, the proximal end of the effecter defines, in the active configuration, a volume throughout which the guide-wire penetrates the intima to perform reentry.

### FIELD OF THE INVENTION

The present invention generally pertains to a device and method for crossing occlusions in vessels of a patient with a guide-wire facilitated in a catheter and more specifically for treating chronic total (arterial) occlusion (CTO).

### BACKGROUND OF THE INVENTION

The present invention relates to Chronic Total Occlusions (CTO's) and especially in arteries. Chronic total occlusions in arteries, both in coronary and peripheral arteries (non-coronary), constitute the most common reason for failure of endovascular (percutaneous, trans-catheter) treatment and referral to open surgery. These arterial lesions constitute the biggest and most important challenge in endovascular therapy. A prerequisite to endovascular treatment of these lesions is the crossing of the occlusions with a guide-wire (from patent lumen on one side to the other) on which therapeutic interventions such as balloon dilation, stent placement and other interventions can be performed. In order to achieve this crossing consistently, reliably and safely, new devices and instrumentation are needed.

The most common cause of arterial occlusive disease is atherosclerosis. Atherosclerosis is a chronic, progressive disease of the arteries in which “plaques” made up of cholesterol deposits, calcium, and abnormal cells develop on the inner lining of the arteries.

Chronic total occlusions exist in both coronary and peripheral arteries, but have been histologically characterized mostly in the coronary arteries. It has been shown that their composition is irregular and changes over time. Specifically, the plaque is not homogenous and contains regions of different composition and structure with multiple neovascular channels.

Outside the lesion and within the outer layer of the arterial wall (the adventitia), the subintimal plane is a consistent potential space of loosely adherent layers. In an effort to avoid the complications associated with open surgical operations, wire-based devices for endovascular treatment of CTO have been introduced. Traditionally, it has been accepted that staying within the lumen and the plaque, rather than outside it, is preferred, so that when the guide-wire exits the occlusion, it is within the arterial lumen beyond it. However, traversing the plaque, which may be hard and calcified, is often difficult even with the use special high-end guide-wires with very stiff tips. To increase the success rate, several devices have been or are being developed. These include among others the Frontrunner (Cordis, J&J) which is based on blunt microdissection, The Crosser (Flowcardia Sunnyvale Calif.) a guide-wire with vibrational energy, the Safe-cross (Intraluminal Therapeutics, Carlsbad Calif.) wire with radiofrequency energy and OCT guidance, the Excimer laser catheter (Spectranetics, Colorado Springs Colo.), CTOS oscillating guide-wire (ReVascular therapeutics Sunnyvale Calif.), CiTop guide-wire (Ovalum Medical Vision, Rehovot Israel) with steerable J-tip guide-wire control and the Niobe system for manipulating instrumentation within magnetic fields. In addition, pharmaco-therapy with local injection of collagenase (Bradley Strauss, Toronto) is being investigated.

### Subintimal Angioplasty

An important endovascular technique to overcome total occlusions is subintimal angioplasty, where a dissection plane is intentionally developed beside the occlusion in the subintimal plane (outside the natural arterial lumen and outside the atherosclerotic plaque, outside the intima and inside the adventitia). This plane is entered from the lumen proximal to the occlusion and the device (wire or catheter) within it is advanced to beyond the occlusion. At this point and preferably not too far away from the termination of the occlusion, the lumen has to be re-entered.

The appeal of the subintimal route is that it is always present, irrespective of the structure and composition of the atherosclerotic plaque. It may be difficult to enter this plane with a guide-wire. This requires puncturing the intima at some point proximal to the occlusion to reach this plane from the arterial lumen. Once inside this plane with a guide-wire, the wire can usually be advanced with a relatively low resistance. The most difficult and least controlled step in this procedure is the reentry. This last step is occasionally difficult and if the guide-wire does not re-enter the true lumen close to the occlusion the entire endovascular procedure may have to be abandoned. Once the guide-wire has entered the lumen beyond the occlusion, the intervention proceeds as usual with balloon dilation and stent application, as required.

Three devices which have been designed to control the re-entry site are on the market. Two devices are similar and use a curved hollow needle, which is driven off center (sideways), to perforate the intima and enter the lumen from the subintimal plane at the desired location. Through this needle, a guide-wire is advanced to the distal lumen, and from this point on the procedure can proceed as usual.

One device is the Outback reentry catheter by Cordis J&J and the other is the Pioneer catheter by Medtronic. Both catheters have a large profile (Outback 6F, Pioneer 7F) which is a disadvantage, especially for use in smaller arteries. Both catheters require precise rotational orientation, so that the needle is driven into the true lumen and not outside the adventitia and the artery. The outback relies on fluoroscopy and a radio-opaque rotation marker, the Pioneer incorporates an IVUS (intravascular ultrasound) catheter for orientation. A third device is a reentry balloon catheter where a flat low profile balloon helps orient the wire towards the lumen (Bridgepoint medical, Minneapolis, Minn.)

Crossing of a CTO in practice: While techniques of crossing the occlusion within the plaque (i.e. intra-luminally) and subintimal angioplasty are presented above as two profoundly different procedures, they are in essence quite similar. In fact, it is occasionally difficult to judge along which plane the guide-wire has actually crossed the total occlusion, whether within the plaque or in the subintimal plane.

The interior of the occluding plaque is inhomogeneous in terms of composition and structure and even within the subintimal plane there may be varying resistance to guide-wire advancement as well. Therefore a means to enhance passage of a guide-wire, to the desired site of reentry, may be helpful.

It is therefore a long felt need to provide a device and method especially adapted to cross occlusions. The device will enable reentry of the guide-wire from the subintimal space into vessel and will also stabilize the tip of the device for advancement of a wire within the lesion.

### SUMMARY OF THE INVENTION

It is one object of the present invention to provide a catheter for crossing occlusions in vessels of a patient. The catheter has a proximal end reversibly insertable within the occlusion and a distal opposite end located outside the patient. The catheter is characterized by vacuum generating mechanism (VGM), lumen in fluid communication with the VGM, pusher and a guide-wire maneuverable within the lumen. The pusher and/or the guide-wire are adapted to at least partially cross the occlusion while an effective measure of vacuum, provided as at least one fluid pulse or other manner, towards the occlusion.

It is another object of the present invention to provide a catheter which comprises at least one open-bore lumen provided in parallel to the main longitudinal axis of the catheter, having a proximal end located at the proximal end of the catheter, and a distal end located outside the patient. The catheter also comprises at least one vacuum generating mechanism (VGM), being in a fluid communication with the lumen's proximal end. The VGM is adapted for generating an effective measure of vacuum and introducing the vacuum towards the proximal end of the catheter, via a portion or an entire length of the lumen. The catheter also comprises at least one movable pusher, adapted to be reversibly reciprocated, by means of an actuating mechanism, along and around the main longitudinal axis of the catheter. The pusher is locatable between at least two configurations, a CLOSE configuration and an OPEN configuration, in the OPEN configuration, the proximal end of the pusher is intimately anchored within the occlusion. The catheter also comprises at least one guide-wire, accommodated within the lumen, reciprocally maneuvered along and around the main longitudinal axis of the lumen. The guide-wire is adapted for crossing the occlusion. Wherein the pusher, in the OPEN configuration, provides within the vessel a preset void volume barriered by means of the pusher, the proximal end of the catheter and the inner surface of the occlusion. Wherein the VGM applies suction within the occlusion. Further wherein the pusher is stabilized and fixed within the occlusion to enable the guide-wire to cross the occlusion and to anchor it in an effective manner.

It is another object of the present invention to provide the catheter as defined above, adapted to treat patients with CTO conditions.

It is another object of the present invention to provide the catheter as defined above, wherein the actuating mechanism is at least one pushing wire. The pushing wire mechanically connected to the pusher. The pushing wire is maneuvered along and around the main longitude axis of the lumen. The pushing wire is adapted to actuate of the reciprocate movement of the pusher.

It is another object of the present invention to provide the catheter as defined above, wherein the actuating mechanism is at least one pushing wire. The pushing wire mechanically connected to the pusher. The pushing wire is maneuvered along and around the lumen. The pushing wire adapted for control of the movement of the pusher.

It is another object of the present invention to provide the catheter as defined above, wherein the pushing wire is a non-flexible wire.

It is another object of the present invention to provide the catheter as defined above, wherein at least one of the lumen is a pusher-lumen adapted to accommodate the pushing wire.

It is another object of the present invention to provide the catheter as defined above, wherein at least one of the lumen is a suction lumen adapted for fluid communication with the VGM.

It is another object of the present invention to provide the catheter as defined above, wherein at least one of the lumen is a guide-wire-lumen adapted to accommodate the guide-wire.

It is another object of the present invention to provide the catheter as defined above, wherein the pusher is a solid cap having a tapered shape, adapted to facilitate advancing, penetrating, anchoring, piercing, crossing or otherwise actuating of the catheter within the occlusion.

It is another object of the present invention to provide the catheter as defined above, wherein at least a proximal portion of the pusher is a curled wire, characterized by a shape selected from a group consisting of helix, sphere, cone, cylinder, egg-like, and any combination thereof.

It is another object of the present invention to provide the catheter as defined above, the pusher is characterized by shape memory.

It is another object of the present invention to provide the catheter as defined above, wherein the pusher is the continuation of the pushing wire.

It is another object of the present invention to provide the catheter as defined above, wherein the pusher is mechanically integrated with the pushing wire.

It is another object of the present invention to provide the catheter as defined above, wherein the guide-wire is characterized by a flexible tip. The tip is located at the proximal end of the guide-wire.

It is another object of the present invention to provide the catheter as defined above, wherein the guide-wire is characterized by a stiff tip. The tip is located at the proximal end of the guide-wire.

It is another object of the present invention to provide the catheter as defined above, wherein the guide-wire is an elongated straight member.

It is another object of the present invention to provide the catheter as defined above, wherein the guide-wire is characterized by a bent tip. The tip is located at the proximal end of the guide-wire.

It is another object of the present invention to provide the catheter as defined above, wherein the guide-wire is characterized by a sharpened tip adapted for puncturetion of the occlusion and of the layers of the vessel.

It is still an object of the present invention to provide a method for crossing an occlusion in vessels of a patient. The method comprises steps selected inter alia from: Providing at least one catheter adapted to cross occlusions in vessels of a patient having a proximal end reversibly insertable within the occlusion and a distal opposite end located outside the patient. The catheter also comprises at least one open-bore lumen provided in parallel to the main longitudinal axis of the catheter, having a proximal end located at the proximal end of the catheter, and a distal end located outside the patient. The catheter also comprises at least one vacuum generating mechanism (VGM), being in a fluid communication with the lumen's proximal end. The VGM is adapted for generating an effective measure of vacuum and introducing the vacuum towards the proximal end of the catheter, via a portion or an entire length of the lumen. The catheter also comprises at least one movable pusher; adapted to be reversibly reciprocate, by means of an actuating mechanism, along and around the main longitudinal axis of the catheter. The pusher is locatable between at least two configurations, a CLOSE configuration and an OPEN configuration, in the OPEN configuration, the proximal end of the pusher is intimately anchored within the occlusion. The pusher, in the OPEN configuration, provides within the vessel a preset void volume barriered by means of the pusher, the proximal end of the catheter and the inner surface of the occlusion. The catheter also comprises at least one guide-wire, accommodated within the lumen, reciprocally maneuvered along and around the main longitudinal axis of the lumen. The guide-wire is adapted for crossing the occlusion; Crossing the occlusion with the guide-wire or otherwise inserting the catheter to a location adjacent to the occlusion; Bringing the pusher into the OPEN configuration by moving the pusher forward, or alternatively, by moving the catheter backward while keeping the pusher adjacent to the occlusion; Generating vacuum by the VGM and applying suction within the occlusion. In this position, the pusher is stabilized and fixed to the inner surface of the occlusion and/or to the inner surface of the vessel to enable the guide-wire to cross the occlusion; Advancing the guide-wire through the lumen into the occlusion while the suction is applied and crossing the occlusion with the guide-wire; Advancing the catheter through the occlusion; In case of subintimal passage, once the occlusion has been passed with the catheter, and the catheter is within the vessel wall in subintimal position, verifying the position of the true lumen of the vessel and performing reentry with the guide-wire. if this step was not successful, continuing to step (h); Moving the guide-wire to a direction distal to the occlusion. bringing pusher to the OPEN configuration. creating vacuum by the VGM and applying suction on the inner surface of the occlusion and/or on the inner surface of the vessel through the void volume. In this position, the pusher is stabilized and fixed to the inner surface of the occlusion and/or to the inner surface of the vessel. Puncturating the intima of the vessel. Advancing guide wire either straight or bent in the direction of the lumen in order to puncture the intima (which is fixed to the pusher by suction) of the vessel in order to gain access to the lumen; Once the guide-wire crossed the occlusion, terminating the suction, bringing the pusher into a the CLOSE configuration, the catheter is removed and the procedure continues with the guide-wire. Alternatively, the catheter advances beyond the occlusion for injection and visualization before its removal; and, Repeating at least one of the previous steps if required.

It is still an object of the present invention to provide the method as defined above, further comprising steps of treating patient with CTO conditions.

It is still an object of the present invention to provide the method as defined above, wherein the step of providing the catheter comprises at least one pushing wire; the pushing wire mechanically connected to the pusher.

It is still an object of the present invention to provide the method as defined above, wherein the step of bringing the pusher into the OPEN configuration comprises actuating the reciprocate movement of the pusher by the maneuvering the pushing wire along and around the main longitude axis of the lumen.

It is still an object of the present invention to provide the method as defined above, wherein the pushing wire is a non-flexible wire.

It is still an object of the present invention to provide the method as defined above, wherein the steps of providing the catheter and bringing the pusher into the OPEN configuration, the lumen is a pusher-lumen; the pushing wire is accommodated within the pusher-lumen.

It is still an object of the present invention to provide the method as defined above, wherein the step of providing the catheter and generating vacuum by the VGM, the lumen is a suction lumen fluidly communicating with the VGM.

It is still an object of the present invention to provide the method as defined above, wherein the step of providing the catheter, the at least one of the lumen is a guide-wire-lumen; the guide-wire is accommodated within the guide-wire-lumen.

It is still an object of the present invention to provide the method as defined above, wherein the step of providing the catheter, the pusher is a solid cap having a tapered shape; the cap is advancing, penetrating, anchoring, piercing, crossing or otherwise actuating the catheter within the occlusion.

It is still an object of the present invention to provide the method as defined above, wherein the step of providing the catheter, least a proximal portion of the pusher is a curled wire, characterized by a shape selected from a group consisting of helix, sphere, cone, cylinder, egg-like, and any combination thereof.

It is still an object of the present invention to provide the method as defined above, wherein the pusher is characterized by shape memory.

It is still an object of the present invention to provide the method as defined above, wherein the pusher is mechanically integrated with the pushing wire.

It is still an object of the present invention to provide the method as defined above, wherein the step of providing the catheter, the guide-wire is characterized by a flexible tip; the tip is located at the proximal end of the guide-wire.

It is still an object of the present invention to provide the method as defined above, wherein the step of providing the catheter, the guide-wire is characterized by a stiff tip; the tip is located at the proximal end of the guide-wire.

It is still an object of the present invention to provide the method as defined above, wherein the step of providing the catheter, the guide-wire is an elongated straight member.

It is still an object of the present invention to provide the method as defined above, wherein the step of providing the catheter, the guide-wire is characterized by a bent tip; the tip is located at the proximal end of the guide-wire.

It is still an object of the present invention to provide the method as defined above, wherein the step of providing the catheter, the guide-wire is characterized by a sharpened tip adapted for puncturetion of the occlusion and of the layers of the vessel.

It is still an object of the present invention to provide a catheter. The catheter also comprises at least one fluid pressure-pulse generator adapted to provide at least one fluid pulse with relatively steep potential difference between initiation and termination. The catheter also comprises at least one lumen. The lumen is in communication with the fluid pressure-pulse generator. Wherein the pressure generator is adapted to provide the at least one fluid pulse through the catheter to the occlusion such that the occlusion is crossed.

It is still an object of the present invention to provide a catheter adapted to treat patients with CTO conditions.

It is still an object of the present invention to provide a catheter additionally comprising a movable pusher attached to the proximal end of the catheter that comes into contact with the occlusion.

It is still an object of the present invention to provide a catheter adapted to reverse the generation of pulses, and thereby to act in a suction manner.

It is still an object of the present invention to provide a method for crossing an occlusion in vessels of a patient, comprising:
a. obtaining at least one catheter adapted to cross an occlusion in a hollow tube-like passage. The catheter also comprises at least one fluid pressure-pulse generator adapted to provide at least one fluid pulse with relatively steep potential difference between initiation and termination. The catheter also comprises at least one lumen. The lumen is in communication with the fluid pressure-pulse generator;
b. attempting to cross the occlusion with a regular guide-wire;
c. bringing the tip of the hydraulic wire in contact with the occlusive lesion;
d. applying a primary series of hydraulic impacts; thereby enabling passage of a guide-wire through the occlusion;
e. advancing the wire through the occlusion.
f. It is still an object of the present invention to provide a method as described above, additionally comprising steps of:
   a. delivering a secondary series of hydraulic impacts to create localized disruption of the occluding tissue in order to enable passage of the wire; and,
   b. entering the lumen beyond the occluded segment.

Vacuum generating mechanism (VGM):

It is according to one embodiment of the invention wherein the vacuum generating mechanism produces negative pressure (suction) and can be connected to the catheter. The negative pressure is controllable and a transparent chamber shows whether body fluid (e.g. blood) is removed. The mechanism may have a negative pressure chamber through which the distal end of the catheter may be passed. This chamber has an air-tight sealing around the entry and exit sites of the catheter so that suction may be applied to the catheter which lacks a special connection port. Alternatively, the vacuum generating mechanism may be connected to a suction port in the catheter by conventional means (e.g., luer lock).

It is according to one embodiment of the invention wherein the catheter has an outer diameter of e.g., about 0.035″ to 4F (0.89-1.33 mm). The term ‘about’ refers hereinafter to any value being ±25% of the defined measure.

It is according to another embodiment of the invention wherein the guide-wire has a diameter between e.g., about 0.008″ to about 0.018″.

It is according to another embodiment of the invention wherein the pusher is in a configuration of a curled wire.

It is according to yet another embodiment of the invention wherein the pusher is at least partially made of a shape memory alloy (e.g., Nitinol) with a front segment which when unconstrained, takes on a three dimensional form to occupy space and prevent collapse of tissue into its inner space. One possible shape among many is a helix with a diameter similar to or somewhat larger than that of the suction wire or catheter. Other shapes include a variety of patterns creating a surface contour of a sphere, a cone, a cylinder or an egg-like form. The tip (front end) of the thin preformed spacing wire is a sphere with a diameter which may be larger than of the catheter, or is tightly curled up so it does not have a sharp pointing tip, but rather an atraumatic one.

It is another object of the present invention to provide a catheter for crossing occlusions in vessels of a patient; said catheter is characterized by a main longitudinal axis, said catheter comprising:
a. at least one open-bore type lumen characterized by a distal end and a proximal end, interconnected by a main longitudinal axis, substantially parallel to said main longitudinal axis of said catheter; said distal end is located out side the body of said patient;
b. at least one guide-wire, accommodated within said lumen, reciprocally maneuvered along and around said main longitudinal axis of said lumen; said guide-wire is adapted to penetrate the intima at said occlusion proximal end and to perform reentry at said lumen at said occlusion's distal end so as to by-pass/cross said occlusion;
c. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima to perform reentry;
   wherein at least a portion of said proximal end of said effecter, in said active configuration, protrudes out of said proximal end of said open-bore type lumen and is adapted to at least partially encircle at least part of the outer circumference of said proximal end of said catheter.

It is another object of the present invention to provide a catheter for crossing occlusions in vessels of a patient; said catheter is characterized by a main longitudinal axis, said catheter comprising:
a. at least one open-bore type lumen characterized by a distal end and a proximal end, interconnected by a main longitudinal axis, substantially parallel to said main longitudinal axis of said catheter; said distal end is located out side the body of said patient;
b. at least one guide-wire, accommodated within said lumen, reciprocally maneuvered along and around said main longitudinal axis of said lumen; said guide-wire is adapted to penetrates the intima to perform reentry so as to by-pass/cross said occlusion;
c. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima to perform reentry;
   wherein at least a portion of said proximal end of said effecter, in said active configuration, protrudes out of said proximal end of said open-bore type lumen and is adapted to be at least partially buckled in the direction of said distal end of said lumen whilst at least partially encircling at least part of the outer circumference of said proximal end of said catheter.

It is another object of the present invention to provide the catheters as defined above, wherein said effecter is adapted to prevent collapse of tissue into its inner space.

It is another object of the present invention to provide the catheters as defined above, wherein said at least a portion of said proximal end of said effecter is about 10% of the total length of said effecter.

It is another object of the present invention to provide the catheters as defined above, wherein said at least a portion of said proximal end of said effecter is at the most 10% of the total length of said effecter.

It is another object of the present invention to provide the catheters as defined above, wherein said at least a portion of said proximal end of said effecter is at least 10% of the total length of said effecter.

It is another object of the present invention to provide the catheters as defined above, wherein at least one of said effecters comprises at least two branches, such that reconfiguration of all of said branches from said inactive configuration to said active configuration results in the at least partially encircling at least part of the outer circumference of said proximal end of said catheter.

It is another object of the present invention to provide the catheters as defined above, wherein at least one of said effecters comprises at least two branches, such that reconfiguration of all of said branches from said inactive configuration to said active configuration results in the at least partially buckling of at least one of said branches in the direction of said distal end of said lumen and at least partial encircle of at least part of the outer circumference of said proximal end of said catheter.

It is another object of the present invention to provide the catheters as defined above, wherein reconfiguration from said active configuration to said inactive configuration is operatable by means of an actuating mechanism.

It is another object of the present invention to provide the catheters as defined above, wherein said actuating mechanism is selected from a group selected from application of heat, application of electric current, application of magnetic field, application of mechanic means or any combination thereof.

It is another object of the present invention to provide the catheters as defined above, wherein said actuating mechanism is adapted to expose said effecter out of said lumen.

It is another object of the present invention to provide the catheters as defined above, wherein said actuating mechanism comprises at least one pushing wire; said pushing wire is mechanically connected to said effecter; such that said pushing wire is adapted to actuate said effecter.

It is another object of the present invention to provide the catheters as defined above, wherein said at least one pushing wire is either a non-flexible wire or a flexible wire.

It is another object of the present invention to provide the catheters as defined above, wherein said proximal end of said effecter is diverged in a non single-point assembled manner.

It is another object of the present invention to provide the catheters as defined above, wherein at least a portion of said proximal end of said effecter comprises n dispersed wired branches, where n is an integer greater than 1.

It is another object of the present invention to provide the catheters as defined above, wherein at least a portion of said proximal end of said effecter is characterized by either a symmetric or asymmetric shape with regards to said main longitudinal axis of said lumen.

It is another object of the present invention to provide the catheters as defined above, wherein said inactive configuration of said effecter is constrained by means of said catheter.

It is another object of the present invention to provide the catheters as defined above, wherein said effecter, in said inactive configuration, is maintained within said lumen such that the same is constrained to its shape by mean of said lumen.

It is another object of the present invention to provide the catheters as defined above, wherein the shape of at least a portion of said proximal end of said effecter is selected from a group consisting of a curled wire, a spring-like shape, spiral-like shape and any combination thereof.

It is another object of the present invention to provide the catheters as defined above, wherein the shape of at least a portion of said proximal end of said effecter is selected from a group consisting of a three dimensional configuration, a two dimensional configuration and a combination thereof.

It is another object of the present invention to provide the catheters as defined above, wherein the shape of at least a portion of said proximal end of said effecter is selected from a group consisting of helix, coil, spiral, sphere, cone, cylinder, egg-like, loop-like, ball-like, ovoid-like, globular-like, globe-like, orbicular-like, polygonal having n ribs, n is an integer greater than 2, and any combination thereof.

It is another object of the present invention to provide the catheters as defined above, wherein said effecter is characterized by having a tapered shape, adapted to facilitate advancing, penetrating, anchoring, piercing, crossing or otherwise actuating of the catheter within the occlusion.

It is another object of the present invention to provide the catheters as defined above, wherein said guide-wire is characterized by a sharpened tip adapted to puncture the occlusion and/or of the layers of the vessel.

It is another object of the present invention to provide the catheters as defined above, wherein at least a portion of said proximal end of said effecter comprises n loops substantially parallel to each other, where said n is an integer greater than 1.

It is another object of the present invention to provide the catheters as defined above, wherein at least two of said loops are interconnected to each other in at least one location.

It is another object of the present invention to provide the catheters as defined above, wherein at least a portion of said proximal end of said effecter is characterized by a cross section selected form a group consisting of circular, triangle, rectangular, oval or any combination thereof.

It is another object of the present invention to provide the catheters as defined above, wherein said effecter, in said inactive configuration, is disposed outside of said lumen.

It is another object of the present invention to provide the catheters as defined above, wherein said proximal end of said effecter is adapted to define, in said inactive configuration, a volume throughout which said guide-wire penetrate the intima at said occlusion proximal end and performs reentry at said lumen at said occlusion's distal end thereby crosses said occlusion.

It is another object of the present invention to provide the catheters as defined above, wherein said effecter is adapted to be reversibly moved, by means of an actuating mechanism, along and around said main longitudinal axis of said catheter.

It is another object of the present invention to provide the catheters as defined above, wherein said effecter is characterized by a spring-like mechanical behavior.

It is another object of the present invention to provide the catheters as defined above, wherein said spring is characterized by either a constant or a variant pitch.

It is another object of the present invention to provide the catheters as defined above, wherein said spring comprises n loops, said n is an integer greater than 1.

It is another object of the present invention to provide the catheters as defined above, wherein said loops are characterized by a constant radius, R.

It is another object of the present invention to provide the catheters as defined above, wherein said loops are characterized by either an increasing radius R, decreasing radius R or any combination thereof radius.

It is another object of the present invention to provide the catheters as defined above, wherein said effecter comprises n wires, where n is an integer greater than 1; wherein each of wires is zig-zaged shaped.

It is another object of the present invention to provide the catheters as defined above, wherein said effecter comprises n wires, where n is an integer greater than 1; wherein each of wires is curled shaped.

It is another object of the present invention to provide the catheters as defined above, wherein said catheter further comprises a vacuum generating mechanism (VGM) adapted to stabilize and fix said effecter in said active configuration within said inner surface of said vessel, to enable said guide wire to penetrate the intima at said occlusion proximal end and to perform reentry at said lumen at said occlusion's distal end so as to by-pass/cross said occlusion.

It is another object of the present invention to provide the catheters as defined above, wherein said VGM is being in a fluid communication with said proximal end of said lumen.

It is another object of the present invention to provide the catheters as defined above, wherein said VGM is adapted for generating an effective measure of vacuum and introducing the vacuum towards said proximal end of said catheter, via at least a portion of said lumen.

It is another object of the present invention to provide the catheters as defined above, wherein said VGM is adapted to apply suction within said inner surface of said vessel; such that said effecter is stabilized and fixed within the same to enable said guide-wire to penetrate the intima at said occlusion proximal end and perform reentry at said lumen at said occlusion's distal end so as to by-pass/cross said occlusion y.

It is another object of the present invention to provide the catheters as defined above, wherein said effecter is adapted, in said active configuration, to provide said vessel with a predetermined void volume, defined by means selected from a group consisting of said effecter, said proximal end of said catheter, the inner surface of said occlusion and any combination thereof.

It is another object of the present invention to provide the catheters as defined above, wherein at least a portion of said proximal end of said effecter in said active configuration, is anchored within said inner surface of said vessel so as to enable said guide-wire to penetrate the intima at said occlusion proximal end and perform reentry at said lumen at said occlusion's distal end so as to by-pass/cross said occlusion.

It is another object of the present invention to provide the catheters as defined above, adapted to treat patients with CTO conditions.

It is another object of the present invention to provide the catheters as defined above, wherein at least a portion of said proximal end of said effecter at least partially reversibly insertable within said occlusion.

It is another object of the present invention to provide the catheters as defined above, wherein at least a portion of said effecter is made of materials having shape memory properties.

It is another object of the present invention to provide the catheters as defined above, wherein at least a portion of said effecter is made of material comprising electro active polymers (EAPs).

It is another object of the present invention to provide the catheters as defined above, wherein at least a portion of said effecter comprises electro active polymers (EAPs) of a type selected from a group consisting of dielectric EAPs, ferroelectric polymers, liquid crystalline polymers, ionic EAPs and any mixture thereof.

It is another object of the present invention to provide the catheters as defined above, wherein at least a portion of said effecter comprises shape memory alloy materials selected from a group consisting of copper-based alloy, NiTi-based materials and mixtures thereof.

It is another object of the present invention to provide the catheters as defined above, wherein at least a portion of said effecter is characterized by at least one selected from a group consisting of soft, resilient, rubbery, elastic, flexible and a combination thereof.

It is another object of the present invention to provide the catheters as defined above, wherein said at least one lumen is adapted to accommodate a pushing wire, adapted to activate said effecter.

It is another object of the present invention to provide the catheters as defined above, wherein said at least one lumen is adapted to accommodate said guide wire.

It is another object of the present invention to provide the catheters as defined above, wherein said effecter is mechanically integrated with said pushing wire.

It is another object of the present invention to provide the catheters as defined above, wherein said guide-wire is characterized by a proximal end and a distal end, said proximal end of said guide-wire is characterized by at least one configuration selected from a group consisting of (a) a flexible tip; (b) a stiff tip; (c) a bent tip; (d) a sharpened tip; and any combination thereof.

It is another object of the present invention to provide the catheters as defined above, wherein said guide-wire is an elongated straight member.

It is another object of the present invention to provide the catheters as defined above, further comprising at least one fluid pressure-pulse generator adapted to provide at least one fluid pulse with relatively steep potential difference between initiation and termination phases; through said catheter to said occlusion, such that said occlusion is by-passed/crossed.

It is another object of the present invention to provide the catheters as defined above, wherein said effecter is coupled to the proximal end of said catheter.

It is another object of the present invention to provide a method for crossing occlusions in vessels of a patient; comprising:
a. providing at least one catheter, said catheter is characterized by a main longitudinal axis, said catheter comprising:
   i. at least one open-bore type lumen characterized by a distal end and a proximal end, interconnected by a main longitudinal axis, substantially parallel to said main longitudinal axis of said catheter; said distal end is located out side the body of said patient;
   ii. at least one guide-wire, accommodated within said lumen, reciprocally maneuvered along and around said main longitudinal axis of said lumen; said guide-wire is adapted to by-pass/cross said occlusion by penetrating the intima and performing reentry; and,
   iii. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima and performing reentry;
b. inserting said catheter to said vessel, to a location adjacent to beyond said occlusion;
c. actuating said effecter so as to reconfigure the same into said active configuration;
d. extracting said guide wire out of said lumen;
e. penetrating the intima at said occlusion's proximal end and performing reentry into the lumen at said occlusion distal end by said guide-wire; thereby crossing said occlusion;
wherein said step of actuating said effecter results in (a) the protruding of at least a portion of said proximal end of said effecter out of said lumen; and, (b) at least partially encircling at least part of the outer circumference of said proximal end of said catheter; thereby enabling said guide-wire to penetrate the intima and performing reentry so as to cross said occlusion.

It is another object of the present invention to provide a method for crossing occlusions in vessels of a patient; comprising:
a. providing at least one catheter, said catheter is characterized by a main longitudinal axis, said catheter comprising:
   i. at least one open-bore type lumen characterized by a distal end and a proximal end, interconnected by a main longitudinal axis, substantially parallel to said main longitudinal axis of said catheter; said distal end is located out side the body of said patient;
   ii. at least one guide-wire, accommodated within said lumen, reciprocally maneuvered along and around said main longitudinal axis of said lumen; said guide-wire is adapted to by-pass/cross said occlusion by penetrating the intima and performing reentry; and,
   iii. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima and performing reentry;
b. inserting said catheter to said vessel, to a location adjacent to and beyond said occlusion;
c. actuating said effecter so as to reconfigure the same into said active configuration;
d. extracting said guide wire out of said lumen;
e. penetrating the intima at said occlusion's distal end and performing reentry into the lumen at said occlusion proximal end by said guide-wire; thereby crossing said occlusion;
wherein said step of actuating said effecter results in (a) the protruding of at least a portion of said proximal end of said effecter out of said lumen; and, (b) be at least partially buckled in the direction of said distal end of said lumen whilst at least partially encircling at least part of the outer circumference of said proximal end of said catheter; thereby enabling said guide-wire to penetrate the intima and performing reentry so as to cross said occlusion.

It is another object of the present invention to provide the methods as defined above, further comprises a step of advancing said guide-wire through said lumen.

It is another object of the present invention to provide the methods as defined above, further comprises a step of advancing said catheter through said occlusion.

It is another object of the present invention to provide the methods as defined above, further comprises a step of bringing the effecter into said inactive configuration.

It is another object of the present invention to provide the methods as defined above, further comprises a step of removing said catheter form said patient's vessel, and, optionally maintaining said guide-wire within said vessel.

It is another object of the present invention to provide the methods as defined above, further comprises a step pushing said catheter through said reentry site or said occlusion's proximal end into the lumen at said occlusion distal end for injection and visualization before its removal.

It is another object of the present invention to provide the methods as defined above, further comprises a step of performing reentry with said guide-wire while applying a suction mechanism.

It is another object of the present invention to provide the methods as defined above, wherein said at least a portion of said proximal end of said effecter is about 10% of the total length of said effecter.

It is another object of the present invention to provide the methods as defined above, wherein said at least a portion of said proximal end of said effecter is at the most 10% of the total length of said effecter.

It is another object of the present invention to provide the methods as defined above, wherein said at least a portion of said proximal end of said effecter is at least 10% of the total length of said effecter.

It is another object of the present invention to provide the methods as defined above, further comprises step of providing at least one of said effecters with at least two branches, such that reconfiguration of all of said branches from said inactive configuration to said active configuration results in the at least partially encircling at least part of the outer circumference of said proximal end of said catheter.

It is another object of the present invention to provide the methods as defined above, further comprises step of providing at least one of said effecters with at least two branches, such that reconfiguration of all of said branches from said inactive configuration to said active configuration results in at least partially buckling of at least one of said branches in the direction of said distal end of said lumen and at least partial encircle of at least part of the outer circumference of said proximal end of said catheter.

It is another object of the present invention to provide the methods as defined above, wherein reconfiguration from said active configuration to said inactive configuration is operatable by means of an actuating mechanism.

It is another object of the present invention to provide the methods as defined above, wherein said actuating mechanism is selected from a group selected from application of heat, application of electric current, application of magnetic field, application of mechanic means or any combination thereof.

It is another object of the present invention to provide the methods as defined above, wherein said actuating mechanism is adapted to expose said effecter out of said lumen.

It is another object of the present invention to provide the methods as defined above, wherein said actuating mechanism comprises at least one pushing wire; said pushing wire is mechanically connected to said effecter; such that said pushing wire is adapted to actuate said effecter.

It is another object of the present invention to provide the methods as defined above, wherein said at least one pushing wire is either a non-flexible wire or a flexible wire.

It is another object of the present invention to provide the methods as defined above, wherein said proximal end of said effecter is diverged in a non single-point assembled manner.

It is another object of the present invention to provide the methods as defined above, wherein at least a portion of said proximal end of said effecter comprises n dispersed wired branches, where n is an integer greater than 1.

It is another object of the present invention to provide the methods as defined above, wherein at least a portion of said proximal end of said effecter is characterized by either a symmetric or asymmetric shape with regards to said main longitudinal axis of said lumen.

It is another object of the present invention to provide the methods as defined above, wherein said inactive configuration of said effecter is characterized by a constrained shape.

It is another object of the present invention to provide the methods as defined above, wherein said effecter, in said inactive configuration, is maintained within said lumen such that the same is constrained to its shape by mean of said lumen.

It is another object of the present invention to provide the methods as defined above, wherein the shape of at least a portion of said proximal end of said effecter is selected from a group consisting of a curled wire, a spring-like shape, spiral-like shape and any combination thereof.

It is another object of the present invention to provide the methods as defined above, wherein the shape of at least a portion of said proximal end of said effecter is selected from a group consisting of a three dimensional configuration, a two dimensional configuration and a combination thereof.

It is another object of the present invention to provide the methods as defined above, wherein the shape of at least a portion of said proximal end of said effecter is selected from a group consisting of helix, coil, spiral, sphere, cone, cylinder, egg-like, loop-like, ball-like, ovoid-like, globular-like, globe-like, orbicular-like, polygonal having n ribs, n is an integer greater than 2, and any combination thereof.

It is another object of the present invention to provide the methods as defined above, wherein at least a portion of said proximal end of said effecter comprises n loops substantially parallel to each other, where said n is an integer greater than 1.

It is another object of the present invention to provide the methods as defined above, wherein at least two of said loops are interconnected to each other in at least one location.

It is another object of the present invention to provide the methods as defined above, wherein at least a portion of said proximal end of said effecter is characterized by a cross section selected form a group consisting of circular, triangle, rectangular, oval or any combination thereof.

It is another object of the present invention to provide the methods as defined above, wherein said effecter, in said inactive configuration, is disposed outside of said lumen.

It is another object of the present invention to provide the methods as defined above, wherein said proximal end of said effecter is adapted to define, in said inactive configuration, a volume throughout which said guide-wire penetrates the inner layer of the vessel wall so as to by-pass/cross said occlusion.

It is another object of the present invention to provide the methods as defined above, wherein said effecter is adapted to be reversibly moved, by means of an actuating mechanism, along and around said main longitudinal axis of said catheter.

It is another object of the present invention to provide the methods as defined above, wherein said effecter is characterized by a spring-like mechanical behavior.

It is another object of the present invention to provide the methods as defined above, wherein said spring is characterized by either a constant or a variant pitch.

It is another object of the present invention to provide the methods as defined above, wherein said spring comprises n loops, said n is an integer greater than 1.

It is another object of the present invention to provide the methods as defined above, wherein said loops are characterized by a constant radius, R.

It is another object of the present invention to provide the methods as defined above, wherein said loops are characterized by a either an increasing, decreasing radius or any combination thereof.

It is another object of the present invention to provide the methods as defined above, wherein said effecter comprises n wires, where n is an integer greater than 1; wherein each of wires is zig-zaged shaped.

It is another object of the present invention to provide the methods as defined above, wherein said effecter comprises n wires, where n is an integer greater than 1; wherein each of wires is curled shaped.

It is another object of the present invention to provide the methods as defined above, wherein said catheter further comprises a vacuum generating mechanism (VGM) adapted to stabilize and fix said effecter in said active configuration within said inner surface of said vessel, to enable said guide wire to reenter said lumen distally to the occlusion.

It is another object of the present invention to provide the methods as defined above, wherein said VGM is being in a fluid communication with said proximal end of said lumen.

It is another object of the present invention to provide the methods as defined above, wherein said VGM is adapted for generating an effective measure of vacuum and introducing the vacuum towards said proximal end of said catheter, via at least a portion of said lumen.

It is another object of the present invention to provide the methods as defined above, wherein said VGM is adapted to apply suction within said inner surface of said vessel; such that said effecter is stabilized and fixed within the same to enable said guide-wire to puncture the intima of said vessel by-pass/cross said occlusion and to.

It is another object of the present invention to provide the methods as defined above, wherein said effecter is adapted, in said active configuration, to provide said vessel with a predetermined void volume, defined by means selected from a group consisting of said effecter, said proximal end of said catheter, the inner surface of said occlusion and any combination thereof.

It is another object of the present invention to provide the methods as defined above, wherein at least a portion of said proximal end of said effecter in said active configuration, is anchored within said inner surface of said vessel so as to enable said guide-wire to penetrates the intima to perform reentry so as to by-pass/cross said occlusion and to reenter the same.

It is another object of the present invention to provide the methods as defined above, adapted to treat patients with CTO conditions.

It is another object of the present invention to provide the methods as defined above, wherein at least a portion of said proximal end of said effecter at least partially reversibly insertable within said occlusion.

It is another object of the present invention to provide the methods as defined above, wherein at least a portion of said effecter is made of materials having shape memory properties.

It is another object of the present invention to provide the methods as defined above, wherein at least a portion of said effecter is made of material comprising electro active polymers (EAPs).

It is another object of the present invention to provide the methods as defined above, wherein at least a portion of said effecter comprises electro active polymers (EAPs) of a type selected from a group consisting of dielectric EAPs, ferroelectric polymers, liquid crystalline polymers, ionic EAPs and any mixture thereof.

It is another object of the present invention to provide the methods as defined above, wherein at least a portion of said effecter comprises shape memory alloy materials selected from a group consisting of copper-based alloy, NiTi-based materials and mixtures thereof.

It is another object of the present invention to provide the methods as defined above, wherein at least a portion of said effecter is characterized by at least one selected from a group consisting of soft, resilient, rubbery, elastic, flexible and a combination thereof.

It is another object of the present invention to provide the methods as defined above, wherein said at least one lumen is adapted to accommodate a pushing wire, adapted to activate said effecter.

It is another object of the present invention to provide the methods as defined above, wherein said at least one lumen is adapted to accommodate said guide wire.

It is another object of the present invention to provide the methods as defined above, wherein said effecter is mechanically integrated with said pushing wire.

It is another object of the present invention to provide the methods as defined above, wherein said guide-wire is characterized by a proximal end and a distal end, said proximal end of said guide-wire is characterized by at least one configuration selected from a group consisting of (a) a flexible tip; (b) a stiff tip; (c) a bent tip; (d) a sharpened tip; and any combination thereof.

It is another object of the present invention to provide the methods as defined above, wherein said guide-wire is an elongated straight member.

It is another object of the present invention to provide the methods as defined above, further comprising at least one fluid pressure-pulse generator adapted to provide at least one fluid pulse with relatively steep potential difference between initiation and termination phases; through said catheter to said occlusion, such that said occlusion is by-passed/crossed.

It is another object of the present invention to provide the methods as defined above, further comprising step of coupling said effecter to the proximal end of said catheter.

It is another object of the present invention to provide the methods as defined above, further comprising step of performing reentry with said guide-wire.

It is another object of the present invention to provide the methods as defined above, further comprising step of puncturating the intima of said vessel.

It is another object of the present invention to provide the methods as defined above, further comprising step of advancing guide wire either straight or bent tip in the direction of the lumen in order to puncture the intima of said vessel in order to gain access to the lumen.

It is another object of the present invention to provide the methods as defined above, further comprising step of removing said catheter and continuing the procedure with said guide-wire.

It is another object of the present invention to provide the methods as defined above, wherein said step of activating said effecter further comprises a step of actuating by a reciprocate movement of said pusher by maneuvering said pushing wire along and around the main longitude axis of said at least one lumen.

It is another object of the present invention to provide the methods as defined above, wherein said step guide-wire is characterized by a sharpened tip adapted to puncture said occlusion and of the layers of said vessel.

It is another object of the present invention to provide the methods as defined above, wherein said effecter is adapted to prevent collapse of tissue into its inner space.

It is still an object of the present invention to provide the methods as defined above, wherein said effecter is characterized by having a tapered shape, adapted to facilitate advancing, penetrating, anchoring, piercing, crossing or otherwise actuating of the catheter within the occlusion.

It is lastly an object of the present invention to provide the methods as defined above, wherein said guide-wire is characterized by a sharpened tip adapted to puncture said occlusion and of the layers of the vessel.

### BRIEF DESCRIPTION OF THE FIGURES

In order to better understand the invention and its implementation in a practice, a plurality of embodiments will now be described, be way of non-limiting example only, with reference to the accompanying drawings, in which

FIG. 1 illustrates a catheter with an external diameter of approximately 0.8-1.5 mm, which has one or more lumens along all or part of its length;

FIG. 2 illustrates another embodiment of the catheter from FIG. 1;

FIG. 3 illustrates the catheter as illustrated in FIG. 1 with a pusher. The pusher in this figure is a wire which has an unconstrained configuration of a helix;

FIG. 4 illustrates the catheter 1 as illustrated in FIG. 3 with a guide-wire having an angled tip;

FIG. 5 illustrates the catheter 1 as illustrated in FIG. 4 with a guide-wire having a straight tip;

FIG. 6 illustrates the catheter as illustrated in FIG. 1 with a pusher and straight guide-wire. The pusher in this figure is a solid cap having a tapered shape. The pusher in this figure is in a CLOSE configuration;

FIG. 7 illustrates the catheter as illustrated in FIG. 6 with a pusher and straight guide-wire. The pusher in this figure is in an OPEN configuration;

FIG. 8 illustrates the catheter as illustrated in FIG. 7 with a pusher a guide-wire. The guide-wire has a rounded tip;

FIG. 9 illustrates wire having an external diameter preferably but not necessarily 0.0035, which preferably but not necessarily has a weighted tip. The tip is preferably straight as shown, or alternatively angled. The lumen ends in a lateral aperture at some distance form the tip;

FIG. 10 illustrates a wire having a lumen which ends in 3 apertures near the tip with channels into apertures pointing from tip backwards;

FIG. 11 illustrates a wire having a lumen within external wall with 3 apertures near the tip and breakable point near the back end to which a tube connectable to a tube connector (such as a luer lock) is attached. Once the back end with the tube is broken off, a rod with a widened tip may be inserted into the lumen of the wire from the back to make the wire useful for passing catheters or other endovascular gear over it;

FIG. 12 illustrates a wire having a lumen within external wall 2 that ends in 3 apertures near the tip and ends with a lateral aperture near the back end where a sealed connection (too small for a screw connection) may be made with an attached tube or the aperture is contained within a high-pressure chamber in the pressure generator;

FIG. 13 illustrates a wire having a lumen with expandable tip at resting position. The tip is attached with springs to the shaft of the wire. At the lower portion of the movable tip there are apertures connected to the central lumen below the tip and covered within the front end of the shaft of the wire;

FIG. 14 illustrates a wire having a lumen with expandable tip with in expanded position. The tip which is attached with springs to the shaft of the wire, is pushed forward and the apertures, which in this drawing point backwards but may point sideways, are exposed beyond the walls of the shaft;

FIG. 15 illustrates a wire having a lumen with movable element. In the wire, 3 apertures near the tip and one aperture some distance back. Movable element is within the wire, has an aperture and a rod running through the wire so that it can be manipulated from the back end. In the position shown, apertures near the tip are covered and aperture some distance back is open through aperture in movable element;

FIG. 16 illustrates the same wire as in FIG. 15 with movable element pulled back so that back aperture is occluded (not aligned with aperture in movable element) and front apertures are open;

FIG. 17 illustrates catheter with lumen. Catheter over the wire (e.g. 3F), with a special channel and lumen for a thin wire (e.g. 0.014) and a separate lumen around the channel of the wire and within the catheter, ending near the front end with 3 apertures, close to the tip and the exit of the wire. The back end of the lumen exits sideways and ends in a connectable tube while the wire goes along the catheter to the back end; and,

FIG. 18 illustrates same catheter as in FIG. 17 but with rapid exchange configuration and exit of wire at mid-shaft.

FIG. 19 schematically illustrates a catheter comprising an effecter with dispersed wired arms active configuration, according to certain embodiments of the present invention;

FIG. 20A schematically illustrates a catheter comprising an effecter with a coiled shape active configuration; according to certain embodiments of the present invention;

FIG. 20B schematically illustrates a catheter comprising two effecters with a spring-like shaped active configuration; according to an embodiment of the present invention;

FIG. 20C schematically illustrates a catheter comprising three effecters with a spring-like shaped active configuration; according to certain embodiments of the invention;

FIG. 21A schematically illustrates a catheter comprising an effecter with separated zig-zag shaped wires, according to some embodiments of the present invention;

FIG. 21B schematically illustrates a top view of the effecter presented in FIG. 21A.

FIG. 22 schematically illustrates an alternative active configuration of the effecter having loops interconnected in several locations, according to some preferred embodiments of the invention.

FIG. 23A schematically illustrates a catheter comprising an effecter having a helix-like active configuration, according to certain embodiments of the invention;

FIG. 23B schematically illustrates a preferred embodiment of the catheter of FIG. 23A;

FIG. 24 schematically illustrates a catheter comprising an effecter with diverged arms, according to some embodiments of the invention; and,

FIG. 25 schematically illustrates a catheter comprising an effecter comprising separate zig-zag shaped wires, constructing a hollow spherical like active configuration, according to certain embodiments of the invention.

### DETAILED DESCRIPTION OF THE PREFERRED EMBODIMENTS

According to one embodiment of the invention, a catheter is provided. The catheter is adapted to cross occlusions in vessels of a patient. The catheter is having a proximal end reversibly insertable adjacent to the occlusion and a distal opposite end located outside the patient. When the pusher in the OPEN configuration, it provides a catheter which is adapted to cross occlusions and methods thereof.

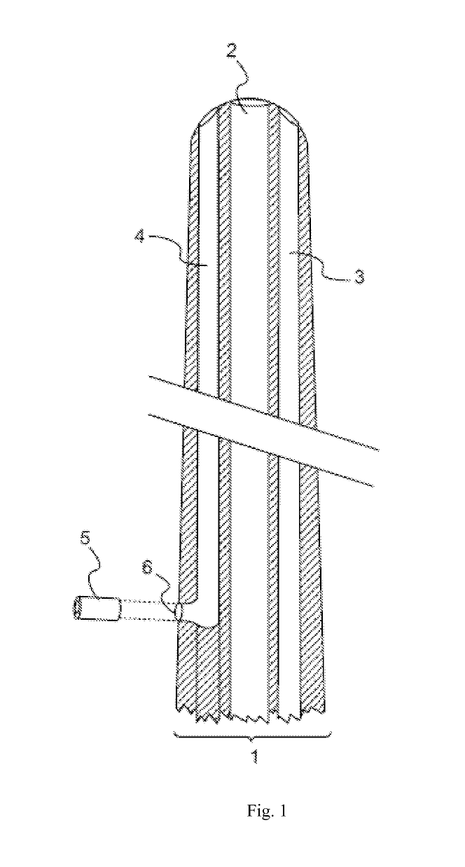

Reference is made now to FIG. 1 where the catheter 1 is schematically shown in an out-of-scale manner. The catheter has e.g., an external diameter of 0.8-1.5 mm, and has a pointed tip for advancing within occlusions. The catheter has a guide-wire lumen 2 which ends at the proximal end of the catheter. This lumen has a diameter of approximately 0.014″. This lumen may be central or eccentric in cross-section of the catheter, The lumen may be round or of a different shape (e.g. oval, cogwheel or other) and is designed to facilitate guiding wire (not shown). There is at least one pushing lumen 3 of another type, terminating beside the tip of the catheter. It is in the scope of the invention wherein the term ‘pushing lumen’ refers to a non-limiting manner to a conduit, pipe, hole, barrel, passageway, facilitated space, or other lumen adapted e.g., by means of size and shape to accommodate the pushing wire. The pushing lumen 3 is designed for accommodation of a pushing wire (not shown) or for the rods of a pusher (not shown). Suction lumen 4 extends along the proximal end of the catheter with an end hole beside end-holes of lumens 2 and 3. The distal end of this lumen is in a fluid communication with the vacuum generating mechanism by tubing and port 5. Alternatively it may end at a side-hole 6 near the distal end of 1 which can be inserted into a suction chamber within the vacuum generating mechanism.

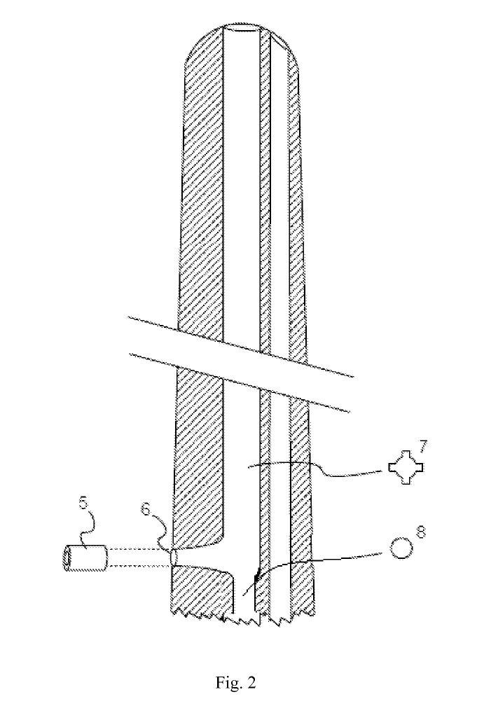

Reference is now made to FIG. 2 which illustrates another embodiment of the catheter schematically shown in an out-of-scale manner. It is in the scope of the invention wherein the term ‘guide-wire lumen’ refers to a non-limiting manner to a conduit, pipe, hole, barrel, passageway, facilitated space, or other lumen adapted e.g., by means of size and shape to accommodate the guide wire. In this embodiment all the components of the catheter are similar to components of the catheter from FIG. 1, and the only difference between the embodiments is that the guide-wire lumen 7 is combined with the suction lumen 8.

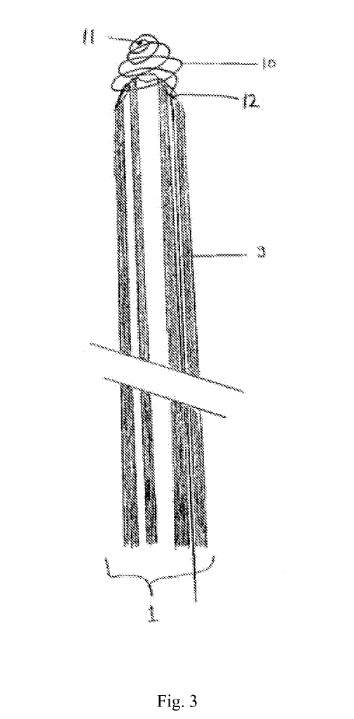

Reference is now made to FIG. 3 which illustrates the catheter 1 schematically shown in an out-of-scale manner, as illustrated in FIG. 1 with a pushing wire 9 in the pushing lumen 12. In this figure, pusher 11 is a wire which has an unconstrained configuration of a helix. The pusher 11 is the continuation of the pushing wire 9. The front tip of the pusher 11 has a sphere connected at the end which can serve to occlude the endhole of the lumen 12 if it is larger than the wire. Alternatively, pusher 11 may be tightly curled up so that it does not have a sharp pointed tip but an atraumatic one.

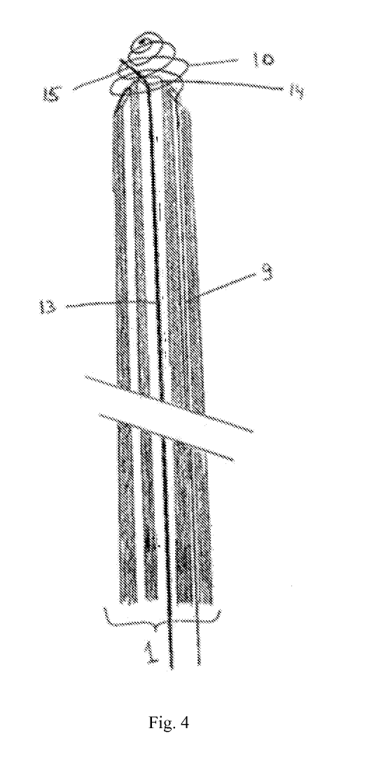

Reference is now made to FIG. 4 which illustrates the catheter 1 schematically shown in an out-of-scale manner, as illustrated in FIG. 3 with a guide-wire 13. The guide-wire 13 is facilitated in the guide-wire lumen 14. The proximal end of the guide-wire 13 has an angled tip 15 and once outside the guide-wire lumen 14 it extends from the sideways of the pusher 10.

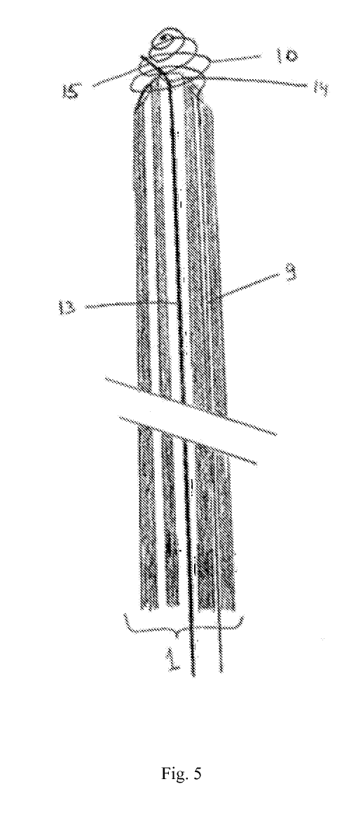

Reference is now made to FIG. 5 which illustrates the catheter 1 schematically shown in an out-of-scale manner, as illustrated in FIG. 4 with a guide-wire 13 that has straight tip 16. The straight tip 16 is extending through the center of the pusher 10.

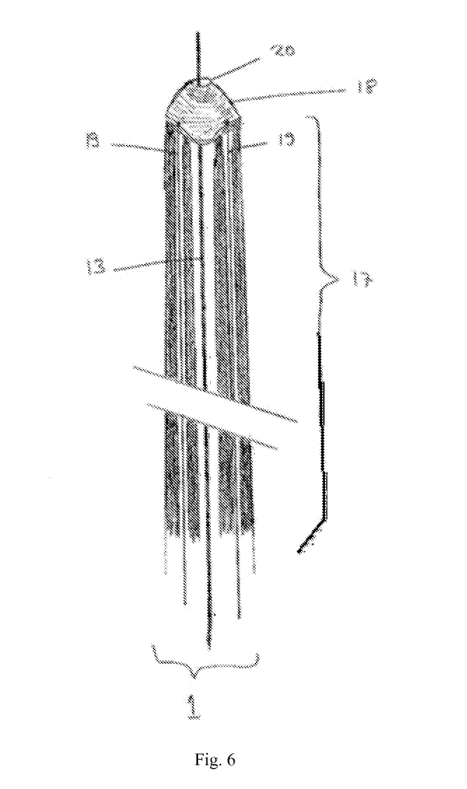

Reference is now made to FIG. 6 which illustrates another embodiment the catheter 1 schematically shown in an out-of-scale manner. In this embodiment the catheter 1 is the same as illustrated in FIG. 1. In this figure pusher 18 is mechanically connected to pushing wires 19 and is in a CLOSE configuration. The pusher 18 is a solid cap having a tapered shape to facilitate advancement of the catheter 1 within the occlusion. A guide-wire 13 is facilitated in the guide-wire lumen 14 and extending through the center of the pusher 18. The suction lumen is not illustrated in this figure but it exists either separately or in conjunction with one or more of the other lumens and terminates at the front surface of the body of the catheter.

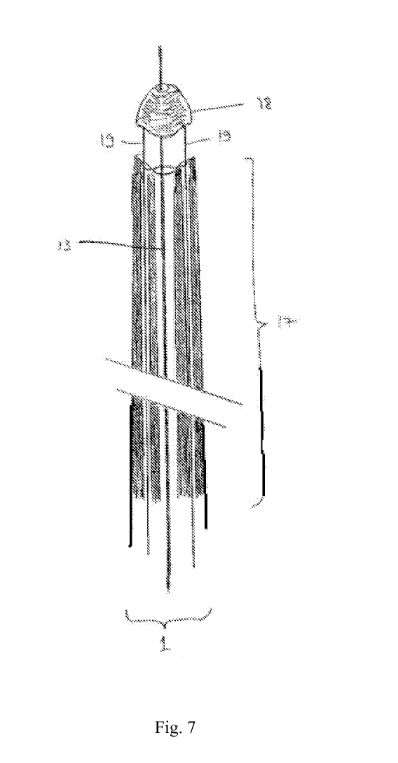

Reference is now made to FIG. 7 which illustrates the catheter 1 schematically shown in an out-of-scale manner, as illustrated in FIG. 6. In this figure all the components are the same, but the pusher 18 is in an OPEN configuration.

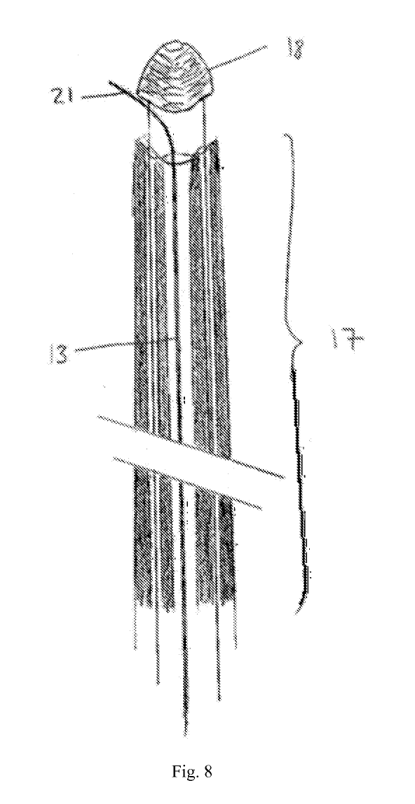

Reference is now made to FIG. 8 which illustrates the catheter 1 as illustrated in FIG. 7 schematically shown in an out-of-scale manner. In this figure all the components are the same, but the guide-wire has a rounded tip 21. The tip 21 is not extending trough the pusher 18, but by-pass it from the side.

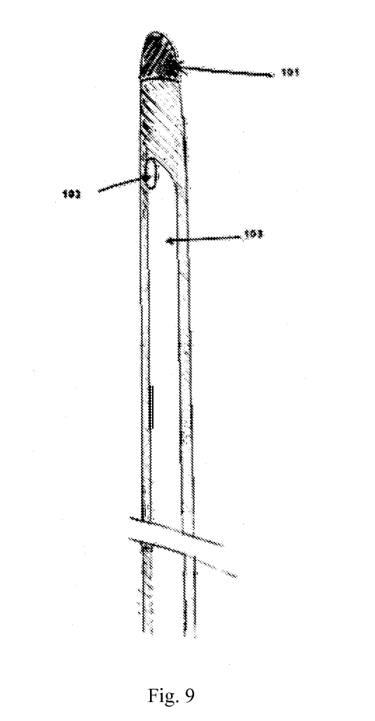

Reference is made now to FIG. 9 illustrating catheter 103 schematically shown in an out-of-scale manner having an external diameter preferably but not necessarily 0.0035, which preferably but not necessarily has a weighted tip 101. The tip is preferably straight as shown, or alternatively angled. The lumen ends in a lateral aperture 102 at some distance form the tip.

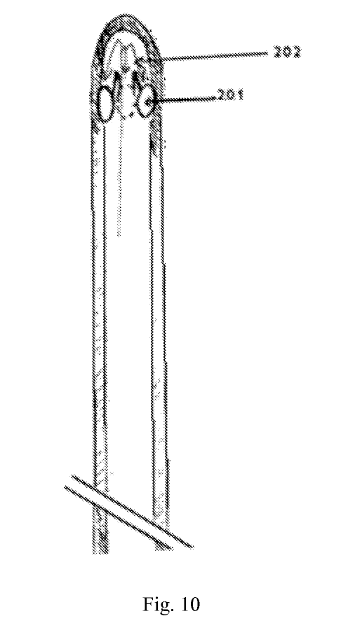

Reference is made now to FIG. 10 illustrating wire with lumen schematically shown in an out-of-scale manner, which ends in 3 apertures near the tip 201 with channels into apertures pointing from tip backwards 202.

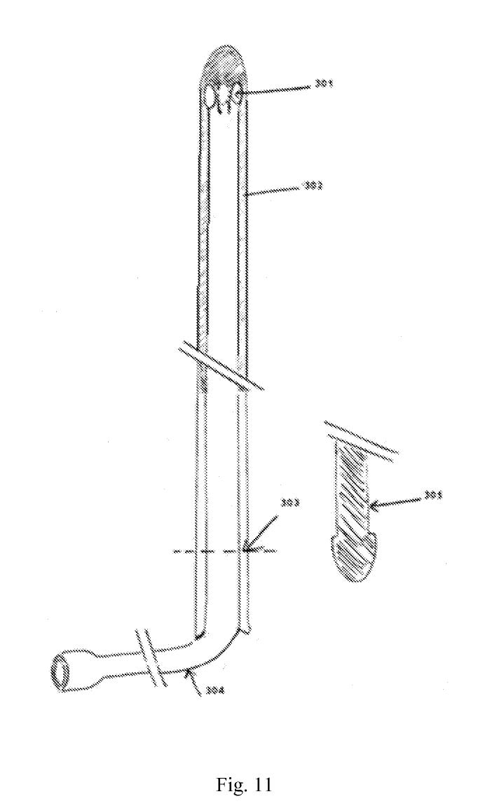

Reference is made now to FIG. 11 illustrating wire with lumen schematically shown in an out-of-scale manner, within external wall 302 with 3 apertures near the tip 301 and breakable point near the back end 303 to which a tube connectable to a tube connector (such as a luer lock) is attached 304. Once the back end with the tube is broken off at 303, a rod with a widened tip 305 may be inserted into the lumen of the wire from the back to make the wire useful for passing catheters or other endovascular gear over it.

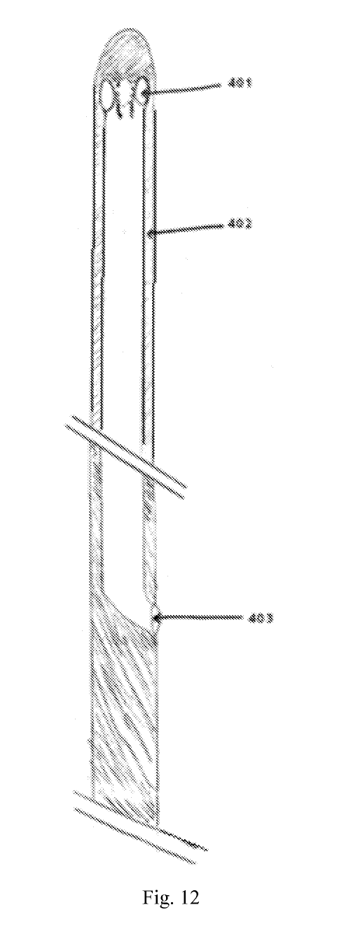

Reference is made now to FIG. 12 illustrating wire with lumen schematically shown in an out-of-scale manner, within external wall 2 that ends in 3 apertures near the tip 401 and ends with a lateral aperture near the back end 403 where a sealed connection (such as with a screw) may be made with an attached tube or the aperture is contained within a high-pressure chamber in the pressure generator.

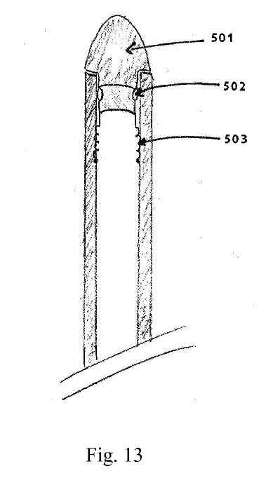

Reference is made now to FIG. 13 illustrating wire with lumen schematically shown in an out-of-scale manner, with expandable tip at resting position. The tip 501 is attached with springs 503 to the shaft of the wire. At the lower portion of the movable tip there are apertures connected to the central lumen below the tip and covered within the front end of the shaft of the wire.

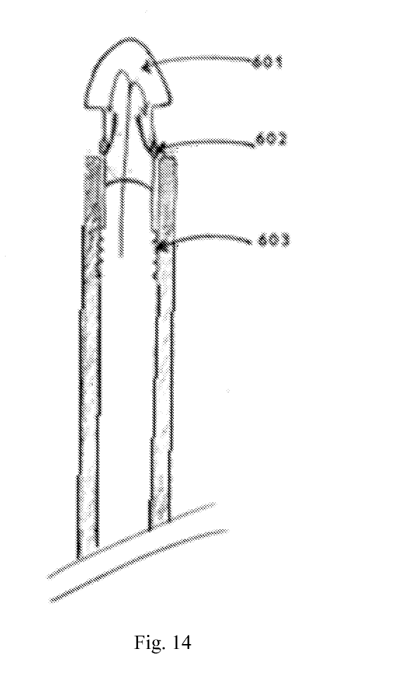

Reference is made now to FIG. 14 illustrating wire with lumen schematically shown in an out-of-scale manner, with expandable tip with in expanded position. The tip 601 which is attached with springs 603 to the shaft of the wire, is pushed forward and the apertures 602, which in this drawing point backwards but may point sideways, are exposed beyond the walls of the shaft.

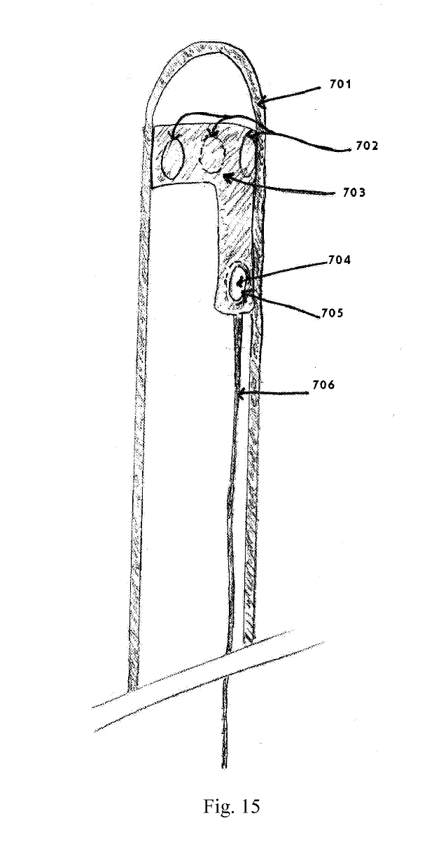

Reference is made now to FIG. 15 illustrating wire with lumen 701 schematically shown in an out-of-scale manner, with movable element 703. In the wire, 3 apertures near the tip 702 and one aperture some distance back 704. Movable element is within the wire, has an aperture 705 and a rod running through the wire 706 so that it can be manipulated from the back end. In the position shown, apertures near the tip 702 are covered and aperture some distance back 704 is open through aperture 705 in movable element.

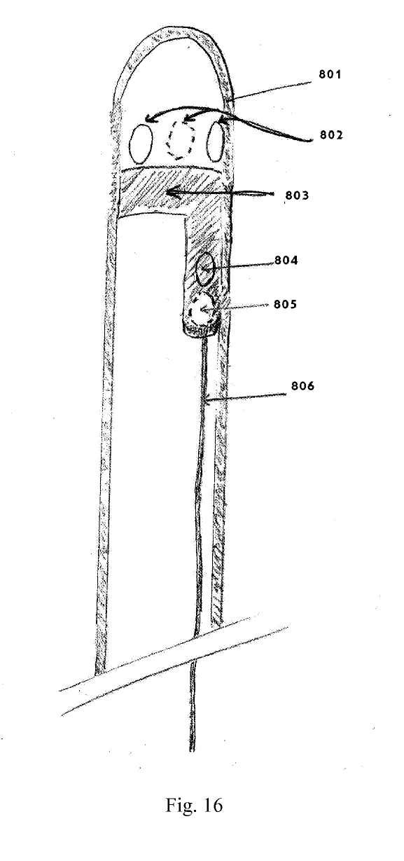

Reference is made now to FIG. 16 illustrating the same wire schematically shown in an out-of-scale manner, as in FIG. 15 with movable element pulled back so that back aperture 804 is occluded (not aligned with aperture 805 in movable element) and front apertures 802 are open.

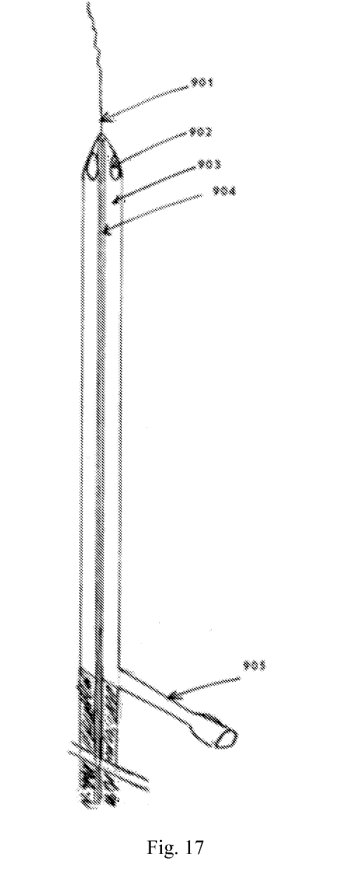

Reference is made now to FIG. 17 illustrating catheter with lumen schematically shown in an out-of-scale manner. Catheter over the wire (e.g. 3F), with a special channel and lumen for a thin wire (e.g. 0.014) 904 and a separate lumen around the channel of the wire and within the catheter 903, ending near the front end with 3 apertures 902, close to the tip and the exit of the wire 901. The back end of the lumen exits sideways and ends in a connectable tube 905 while the wire goes along the catheter to the back end.

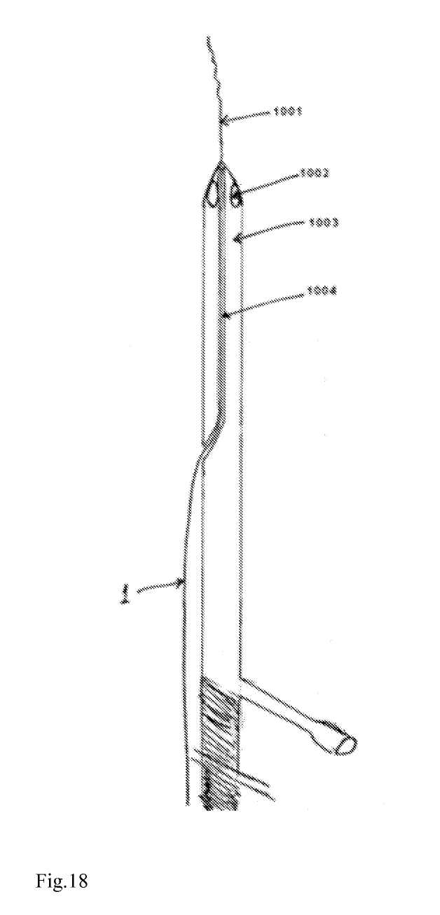

Reference is made now to FIG. 18 illustrating same catheter schematically shown in an out-of-scale manner, as in FIG. 17 but with rapid exchange configuration and exit of wire 1001 at mid-shaft.

The present invention provides a catheter for crossing occlusions in vessels of a patient; said catheter is characterized by a main longitudinal axis, said catheter comprising:
a. at least one open-bore type lumen characterized by a distal end and a proximal end, interconnected by a main longitudinal axis, substantially parallel to said main longitudinal axis of said catheter; said distal end is located out side the body of said patient;
b. at least one guide-wire, accommodated within said lumen, reciprocally maneuvered along and around said main longitudinal axis of said lumen; said guide-wire is adapted to penetrate the intima at said occlusion proximal end and to perform reentry at said lumen at said occlusion's distal end so as to by-pass/cross said occlusion;
c. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima to perform reentry;
   wherein at least a portion of said proximal end of said effecter, in said active configuration, protrudes out of said proximal end of said open-bore type lumen and is adapted to at least partially encircle at least part of the outer circumference of said proximal end of said catheter.

The present invention further a catheter for crossing occlusions in vessels of a patient; said catheter is characterized by a main longitudinal axis, said catheter comprising:
a. at least one open-bore type lumen characterized by a distal end and a proximal end, interconnected by a main longitudinal axis, substantially parallel to said main longitudinal axis of said catheter; said distal end is located out side the body of said patient;
b. at least one guide-wire, accommodated within said lumen, reciprocally maneuvered along and around said main longitudinal axis of said lumen; said guide-wire is adapted to penetrates the intima to perform reentry so as to by-pass/cross said occlusion;
c. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima to perform reentry;
   wherein at least a portion of said proximal end of said effecter, in said active configuration, protrudes out of said proximal end of said open-bore type lumen and is adapted to be at least partially buckled in the direction of said distal end of said lumen whilst at least partially encircling at least part of the outer circumference of said proximal end of said catheter.

It is a main aspect of the invention that the catheter herein disclosed is adapted to treat patients with Chronic Total (arterial) Occlusion (CTO) conditions.

It is also within the scope of the present invention to provide a method for crossing occlusions in vessels of a patient; comprising:
a. providing at least one catheter, said catheter is characterized by a main longitudinal axis, said catheter comprising:
   i. at least one open-bore type lumen characterized by a distal end and a proximal end, interconnected by a main longitudinal axis, substantially parallel to said main longitudinal axis of said catheter; said distal end is located out side the body of said patient;
   ii. at least one guide-wire, accommodated within said lumen, reciprocally maneuvered along and around said main longitudinal axis of said lumen; said guide-wire is adapted to by-pass/cross said occlusion by penetrating the intima and performing reentry; and,
   iii. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima and performing reentry;
b. inserting said catheter to said vessel, to a location adjacent to beyond said occlusion;
c. actuating said effecter so as to reconfigure the same into said active configuration;
d. extracting said guide wire out of said lumen;
e. penetrating the intima at said occlusion's proximal end and performing reentry into the lumen at said occlusion distal end by said guide-wire; thereby crossing said occlusion;
wherein said step of actuating said effecter results in (a) the protruding of at least a portion of said proximal end of said effecter out of said lumen; and, (b) at least partially encircling at least part of the outer circumference of said proximal end of said catheter; thereby enabling said guide-wire to penetrate the intima and performing reentry so as to cross said occlusion.

It is another object of the present invention to provide a method for crossing occlusions in vessels of a patient; comprising:
a. providing at least one catheter, said catheter is characterized by a main longitudinal axis, said catheter comprising:
   i. at least one open-bore type lumen characterized by a distal end and a proximal end, interconnected by a main longitudinal axis, substantially parallel to said main longitudinal axis of said catheter; said distal end is located out side the body of said patient;
   ii. at least one guide-wire, accommodated within said lumen, reciprocally maneuvered along and around said main longitudinal axis of said lumen; said guide-wire is adapted to by-pass/cross said occlusion by penetrating the intima and performing reentry; and,
   iii. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima and performing reentry;
b. inserting said catheter to said vessel, to a location adjacent to and beyond said occlusion;
c. actuating said effecter so as to reconfigure the same into said active configuration;
d. extracting said guide wire out of said lumen;
e. penetrating the intima at said occlusion's distal end and performing reentry into the lumen at said occlusion proximal end by said guide-wire; thereby crossing said occlusion;
wherein said step of actuating said effecter results in (a) the protruding of at least a portion of said proximal end of said effecter out of said lumen; and, (b) be at least partially buckled in the direction of said distal end of said lumen whilst at least partially encircling at least part of the outer circumference of said proximal end of said catheter; thereby enabling said guide-wire to penetrate the intima and performing reentry so as to cross said occlusion.

It is also within the scope of the present invention to provide a method for crossing an occlusion in vessels of a patient. The method comprises steps selected inter alia from: Providing at least one catheter adapted to cross occlusions in vessels of a patient having a proximal end reversibly insertable within the occlusion and a distal opposite end located outside the patient. The catheter also comprises at least one open-bore lumen provided in parallel to the main longitudinal axis of the catheter, having a proximal end located at the proximal end of the catheter, and a distal end located outside the patient.

The catheter may also comprise at least one vacuum generating mechanism (VGM), being in a fluid communication with the lumen's proximal end. The VGM is adapted for generating an effective measure of vacuum and introducing the vacuum towards the proximal end of the catheter, via a portion or an entire length of the lumen.

The catheter also comprises at least one movable effecter; adapted to be reversibly reciprocate, by means of an actuating mechanism, along and around the main longitudinal axis of the catheter.

The effecter is locatable between at least two configurations, a inactive configuration and an active configuration, in the active configuration, the proximal end of the effecter is intimately anchored within the occlusion.

The effecter, in the active configuration, provides within the vessel a preset void volume barriered by means of the effecter, the proximal end of the catheter and the inner surface of the occlusion. The effecter, in the active configuration, protrudes out of said proximal end of said open-bore type lumen and is adapted to at least partially encircle at least part of the outer circumference of said proximal end of said catheter.

The catheter also comprises at least one guide-wire, accommodated within the lumen, reciprocally maneuvered along and around the main longitudinal axis of the lumen.

The guide-wire is adapted for crossing\by-passing the occlusion;

Crossing\by passing the occlusion by the guide-wire is provided by adjacently bringing the catheter to a location as close as possible to the occlusion; Bringing\activating the effecter into the active configuration while keeping the effecter adjacent to the occlusion;

stabilizing and fixing the effecter to the inner surface of the occlusion and/or to the inner surface of the vessel to enable the guide-wire to by-pass the occlusion.

Advancing the guide-wire through the lumen into the occlusion while the suction is applied and crossing/by passing the occlusion with the guide-wire.

Alternatively, vacuum is applied on the inner surface of the occlusion and/or on the inner surface of the vessel.

In this position, the effecter is stabilized and fixed to the inner surface of the occlusion and/or to the inner surface of the vessel. Then, the intima of the vessel is punctured. Next, the guide wire is advanced either straight or bent in the direction of the lumen in order to puncture the intima (which is fixed to the effecter by suction) of the vessel in order to gain access to the lumen;

Once the guide-wire crossed the occlusion, the suction is terminated and the effected is reconfigured into the inactive configuration; the catheter is removed and the procedure continues with the guide-wire.

Alternatively, the catheter advances beyond the occlusion for injection and visualization before its removal; and, repeating at least one of the previous steps if required.

Such effecters when protrudes out of the catheter provides mechanical support for the guide wore and enables the same to penetrate the intima at said occlusion proximal end and to perform reentry at said lumen at said occlusion's distal end so as to by-pass/cross the occlusion.

As used herein, the term “bore type lumen” refers in a non-limiting manner to a conduit, pipe, tunnel, hole, barrel, passageway, facilitated space, or other cavity or channel with a tubular structure, adapted, i.e. by means of size and shape, to accommodate, according to certain embodiments, at least one of the following: at least a portion of the guide-wire, at least a portion of the effecter and at least a portion of an actuating mechanism.

The term “guide wire” used herein refers to a long and flexible fine spring or spring—like member, that may be used to introduce and position an intravascular angiographic catheter, usually used in the Seldinger technique.

The term “effecter” as used herein, refers in a non limiting manner to a member or actuator adapted to cause or achieve a result or to produce an outcome or to accomplish an effect.

According to preferred embodiments of the invention, such an effecter has an active configuration and an inactive configuration. In said active configuration, at least a portion of the at least one effecter is preferably protruding out of the proximal end of the catheter and located within the inner surface of the vessel.

According to one embodiment, in the active configuration, at least a portion of the proximal end of the effecter is preferably adapted, at least partially to encircle, surround, or enclose at least part of the outer circumference or outer rim of the proximal end of the catheter. In another embodiment, the effecter is disposed in front or ahead of the proximal end of the lumen. It is a core aspect of the invention that the proximal end of the effecter is adapted to define or confine a void volume, throughout which the guide-wire by-passes the occlusion.

The term ‘about’ refers hereinafter to any value being ±25% of the defined measure.

The term “branch” used herein refers in a non limiting manner to an arm or limb or part, section or member of the at least one effecter.

The term “active configuration” used herein refers inter alia to an effective or operative disposition, arrangement, design, or structure of the at least one effecter that protrudes out of the proximal end of the catheter. According to main embodiments of the invention the at least one effecter is designed to define a volume within the vessel throughout which the guide-wire by-passes the occlusion. The aforementioned active configuration is further adapted to prevent collapse of the inner tissues of the vessel to the interior space, so as to enable the guide wire to by pass the occlusion.

In one aspect of the invention, the active configuration of the at least one effecter includes a conformation in which at least a portion of the proximal end of the at least one effecter is adapted to at least partially encircle or surround or enclose at least part of the outer circumference or outer rim of the proximal end of the catheter. In a further aspect of the invention, at least a portion of the proximal end of the at least one effecter is adapted to be at least partially buckled towards the distal end of the catheter whilst at least partially encircling at least part of the outer circumference of the proximal end of said catheter. In another aspect of the invention, the proximal end of the at least one effecter, in its active configuration, is diverged in a non single-point assembled manner, such that the proximal end of the effecter is open, or is assembled into a single point positioned at the proximal end of the guide wire such that the proximal end of the at least one effecter is converged to a single point. In both cases the proximal end of the at least one effecter is located adjacent to the occlusion site within the vessel. An active configuration as herein disclosed may preferably include three dimensional conformations comprising loop-like, helix-like and coiled, curled, spiral, spring-like arm-like, branch-like elements, structures or conformations or a combination thereof. It is herein further disclosed that at least a portion of the at least one effecter in its active configuration has a conformation or shape selected from a group consisting of sphere, cone, cylinder, egg-like, loop-like, ball-like, ovoid-like, globular-like, globe-like, orbicular-like, polygonal having n ribs, n is an integer greater than 2, and any combination thereof.

According to certain embodiments of the invention, the effecter conforms or reconfigured into the active configuration by either passively or actively facilitated actuating mechanism.

The term “inactive configuration” used herein refers to a shape or conformation which is non functional or non active. It is herein disclosed that the inactive configuration of the effecter could be at least partially disposed within the lumen and thus the same is constrained by the lumen of the catheter; or at least partially located outside the lumen protruding out of the proximal end of the catheter.

The term “spring—like” used herein refers to an elastic object adapted to store mechanical energy or behavior. It is within the scope of the present invention that a spring like shape may be characterized by a constant or non constant (variant) pitch. In a further embodiment a spring like shape may be characterized by n loops, each of said loops is characterized by a constant radius R or alternatively by a varied radius R which decreases or increases with n.

It is a further main aspect of the invention that at least a portion of the effecter is made of materials having shape memory properties.

The term ‘shape memory properties’ used herein refers to a material or alloy that “remembers” its shape, and can be returned to that shape after being deformed. Shape memory alloys have numerous applications in the medical and aerospace industries. According to certain embodiments of the invention, shape memory alloy materials may include, but are not limited to copper-zinc-aluminum-nickel, copper-aluminum-nickel, and nickel-titanium (NiTi) alloys or any other copper-based alloys, NiTi-based materials and mixtures thereof.

The term “Electroactive Polymers”, or “EAPs” used herein refer to polymers that exhibit a change in size or shape when stimulated by an electric field. EAPs are characterized by the ability to substantially undergo deformation while sustaining large forces. Such materials are commonly used for actuators and sensors applications. It is within the scope of the invention that types of Electroactive Polymers may include, but are not limited to dielectric EAPs, Ferroelectric Polymers, Liquid Crystalline Polymers, Ionic EAPs and mixtures thereof.

The term “Dielectric EAPs” used herein refer to materials in which actuation is caused by electrostatic forces, optionally, between two electrodes which squeeze the polymer. Dielectric elastomers are capable of withstanding very high strains and are adapted to fundamentally being a capacitor that changes its capacitance when a voltage is applied, by allowing the polymer to compress in thickness and expand in area due to the electric field. Examples of Dielectric EAPs may include electrostrictive polymers and dielectric elastomers.

The term “Ferroelectric Polymers” used herein refer to a group of crystalline polar polymers that are also ferroelectric, meaning that they maintain a permanent electric polarization that can be reversed, or switched, in an external electric field. Ferroelectric polymers, such as polyvinylidene fluoride (PVDF), are used in acoustic transducers and electromechanical actuators because of their inherent piezoelectric response, and as heat sensors because of their inherent pyroelectric response.

The term “Liquid Crystalline Polymers” used herein refer to liquid crystalline polymers comprising mesogenic groups linked to each other by a flexible spacer. The mesogens within a backbone form the mesophase structure causing the polymer itself to adopt a conformation compatible with the structure of the mesophase. The direct coupling of the liquid crystalline order with the polymer conformation has given main-chain liquid crystalline elastomers a large amount of interest. The synthesis of highly oriented elastomers leads to have a large strain thermal actuation along the polymer chain direction with temperature variation resulting in unique mechanical properties and potential applications as mechanical actuators

The term “Ionic EAPs” used herein refer to EAPs in which actuation is caused by the displacement of ions inside the polymer. Examples of ionic EAPS are conductive polymers, ionic polymer-metal composites (IPMCs), and responsive gels. Yet another example is a Bucky gel actuator, which is a polymer-supported layer of polyelectrolyte material consisting of an ionic liquid sandwiched between two electrode layers consisting of a gel of ionic liquid containing single-wall carbon nanotubes.

The term “pitch” used herein refers to the distance from the center of one coil to the center of the adjacent coil in a spring or a helix—like shape.

According to certain embodiments of the invention, the aforementioned effecter is operatable between the inactive and active configurations by means of an actuating mechanism, preferably by a source of energy such as electric current, hydraulic pressure, pneumatic pressure, application of heat, application of magnetic field, application of mechanic means or any combination thereof.

According to a further main aspect of the invention, the proximal end of the effecter is diverged in a non single-point assembled manner. Such an open configuration enables the guide wire to pass or protrude through the effecter in order to penetrate the intima at said occlusion proximal end and to perform reentry at said lumen at said occlusion's distal end so as to by-pass/cross said occlusion.

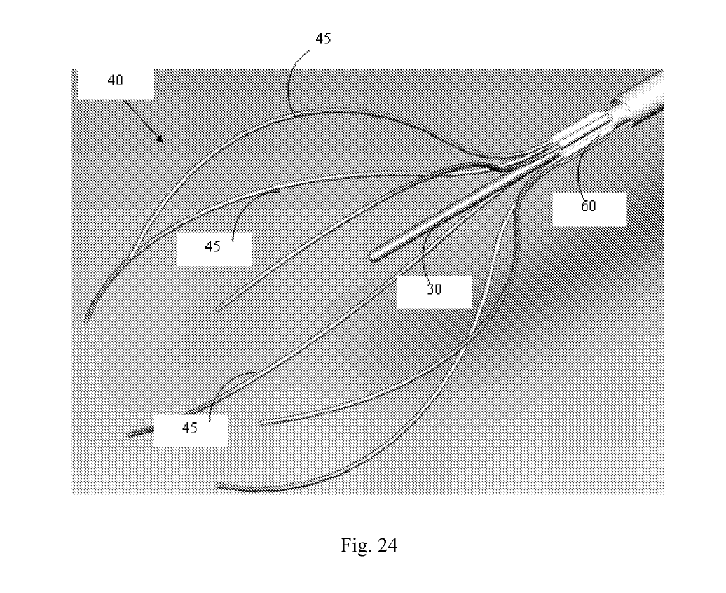

In certain embodiments, the diverged configuration (in which the proximal end of the effecter is diverged into a non single-point assembled manner, see for example FIG. 24a) enables the effecter to reciprocally conform into the inactive configuration (i.e., pulled back into the catheter), while maintaining the guide wire exposed within the inner surface of the vessel.

Reference is now made to the drawings showing preferred embodiments of the catheter having an effecter characterized by alternative active configuration conformations as optional examples of the present invention.

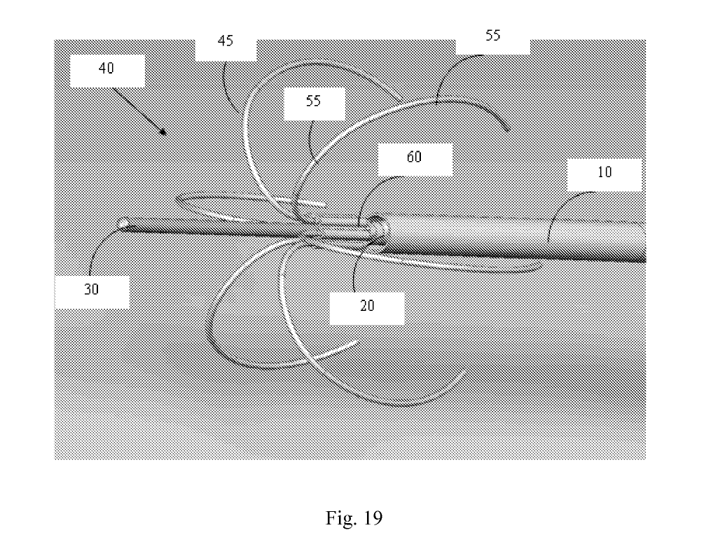

Reference is made now to FIG. 19 schematically illustrating a catheter comprising an effecter 40 with dispersed wired arms 45. The term arm used herein also refers to a branch, a segment or a part of the effecter.

The catheter 10 is adapted to accommodate within its longitudinal lumen 20 a guide-wire 30 and at least a portion of the effecter. In one embodiment, the catheter 10 has e.g., an external diameter of about 0.8-1.5 mm.

The lumen 20 has a diameter of approximately 0.014″.

After insertion of the catheter adjacent to an occlusion within a vessel, the effecter is being reconfigured from the inactive configuration to the active configuration so as to emerge out of the catheter's constrains (namely, the lumen 20).

Thus, by protruding out of the catheter's proximal end, the effecter conforms into its active configuration. In this figure, at least a portion of the proximal end of the effecter comprises n dispersed wired arms 45, wherein n is an integer greater than 1. Specifically, the proximal end of the effecter comprises 6 wired arms 45.

According to certain embodiments of the invention, each of the wired arms comprises more than one segment 55.

In the active configuration, the arms are dispersed in a three dimensional arrangement, in which said three dimensional arrangement at least partially encircles at least a portion of the main longitudinal axis of the catheter.

According to a further embodiment of the invention, the wired arms are buckled/bent backwards towards the distal end of the catheter 10.

It should be pointed out that according to another embodiment of the present invention the wired arms, in the inactive configuration are within the lumen 20 and in the active configuration protrudes out of the lumen.

It should be pointed out that according to another embodiment of the present invention the wired arms, in the inactive configuration protrudes out of the lumen 20 and in the active configuration the wired arms buckle/bent backwards towards the distal end of the catheter 10.

According to another embodiment of the present invention, once the wired arms protrudes out of the lumen 20 they can be either passively buckle/bent backwards or actively buckle/bent backwards.

In the active buckling mode—an actuation mechanism activates the same and transform the wired arms from the initial configuration to the buckled configuration, in which said wired arms at least partially encircles at least a portion of the main longitudinal axis of the catheter.

In this mode the wired arms are made of material with elastic properties, such as shape memory alloys, EAPs or other materials with elastic or flexible elements.

In the passive buckling mode the wired arms are made from the initial configuration to the buckled configuration, in which said wired arms at least partially encircles at least a portion of the main longitudinal axis of the catheter.

In this mode the wired arms are made of material with elastic properties, such as spring-like properties, shape memory alloys, EAPs or other materials with elastic or flexible elements.

If the wired arms are made of, e.g., spring-like material, one the same are extracted from lumen 20, the constrains of the lumen are removed and the wired arms buckle.

Thus it is within the scope of the invention, to provide the effecter made of a flexible separate wires 45 characterized by a spring-like characteristics.

In such a case, the effecter, may be facilitated to reconfigure by, for example, emergence out of the catheter's confines. Thus, said effecter's default configuration is the bent/buckled configuration; however, as long as the effecter is maintained within the catheter (and thus, in the inactive configuration) the catheter constrains the same and prevents the same from buckling.

Once said effecter protrudes out of said catheter, it may reconfigure to it default configuration (to the active configuration).

Alternatively, the emergence of the effecter from the catheter can be facilitated by a pushing member 60, mechanically connected to the distal end of the effecter branches 45.

In another embodiment, the effecter conforms into its active configuration by an active mechanism for example by an actuating means.

As illustrated in FIG. 19, the wired arms of the effecter, in its active configuration are folded or curled backward, such that at least a portion of the proximal ends of the arms encircle the proximal end of the catheter. Such an active configuration is designed to define a volume through which the guide wire 30 can intimately reach the occlusion so as to by pass it successfully.

According to one embodiment, the effecter is made of at least one selected from a group consisting of copper-based alloy, NiTi-based materials, electro active polymers (EAPs and mixtures thereof. Thus, the activating mechanism which transforms the effecter from the active configuration to the inactive configuration and vice versa is selected from a group consisting of application of heat, application of electric current, application of magnetic field, application of mechanic means or any combination thereof.

As mentioned above the main advantages of the present invention is the fact that such effecters when protrudes out of the catheter provides mechanical support for the guide wore and enables the same to penetrate the intima at said occlusion proximal end and to perform reentry at said lumen at said occlusion's distal end so as to by-pass/cross the occlusion.

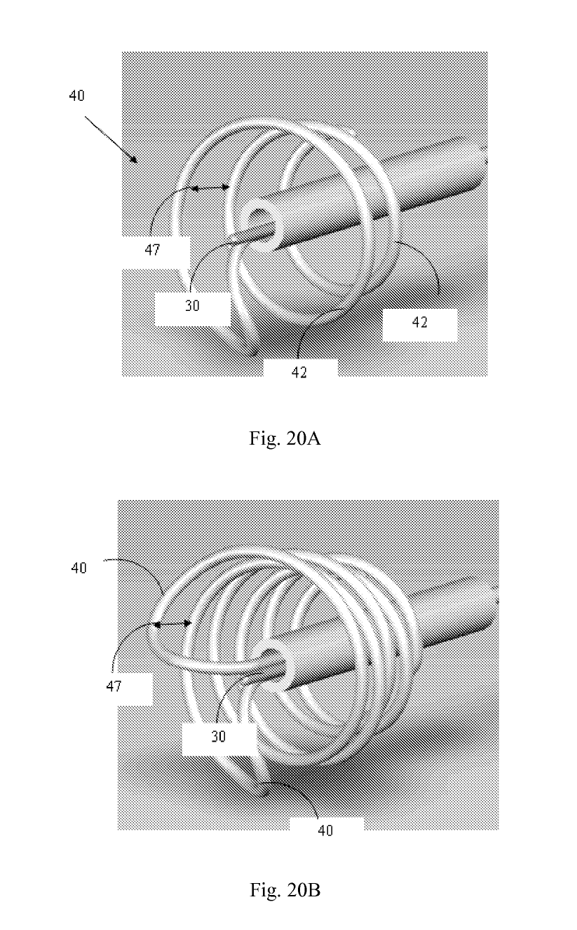

Reference is now made to FIG. 20A schematically illustrating a catheter 10 comprising an effecter 40 with a coiled or spring like shaped wire once the effecter emerge out of the proximal end of the lumen 20, the proximal end of the effecter 40 deforms into a coiled three dimensional conformation.

As shown in this Fig. the effecter is reconfigured to a spring-like or curled shape with two helixes or loop-like structures 42 that encircle at least a portion of the proximal part of the catheter in a symmetrical manner, with respect to the main longitudinal axis of the lumen.

In this configuration, a void volume or space is defined within the vessel, enabling the catheter accommodating the guide-wire 30 to proximally approach the occlusion such that the proximal end of the guide-wire 30 at least partially extends from the proximal end of the lumen 20 and may penetrate the intima at said occlusion proximal end and to perform reentry at said lumen at said occlusion's distal end so as to by-pass/cross said occlusion.

Reference is now made to FIG. 20B schematically illustrating a catheter comprising two effecters with a spring-like shape as in FIG. 20a.

In this Fig., each of the effecters 40 has an unconstrained active configuration of a spiral helix, encircling the proximal part of the outer circumference of the catheter 10.

As illustrated in the Fig. the two effecters are comprised of wires extending from the proximal end of the lumen 20 of the catheter 10 and tightly curled into a spring like configuration encircling the outer rim of the catheter 10 and providing the effecter with a substantially mechanical strength, stabilizing and fixing the effecter in the inner surface of the vessel.

The spring structure of each of the effecters is characterized by either a constant or a variant pitch 47.

In certain embodiments of the invention the spring like configuration comprises n loops 42 characterized by a constant or variable radius R, wherein n is an integer greater than 1.

Alternatively, as shown in FIG. 20C, the catheter may comprise three separate effecters 40 characterized by three separate curled shaped wires protruding from the proximal end of the lumen 20 of the catheter 10 and configured into a tightly coiled spring-like configuration encircling at least a portion of the outer rim of the catheter 10.

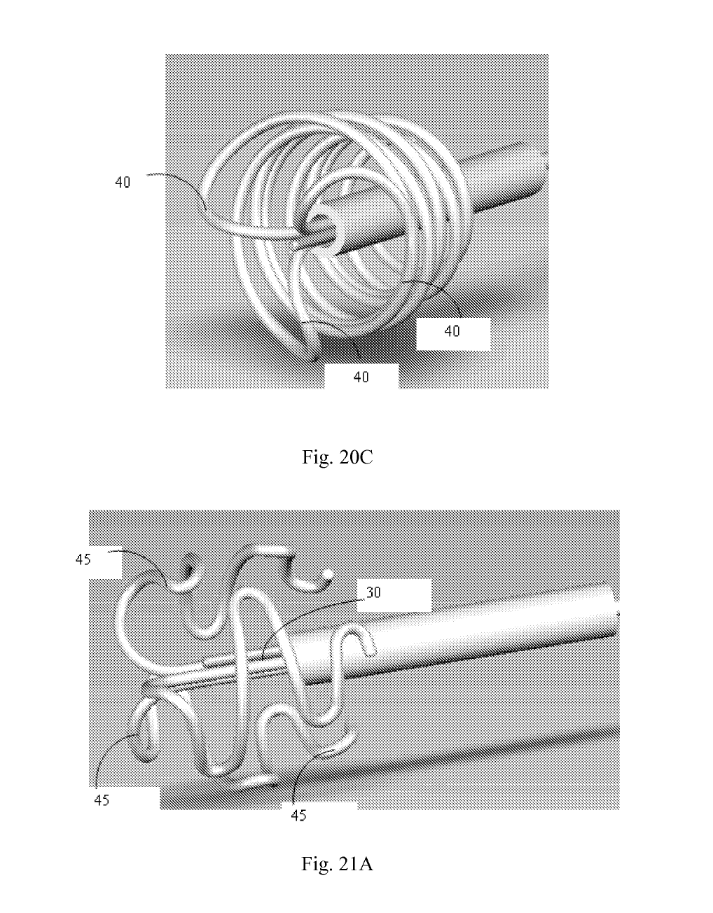

Reference is now made to FIG. 21 schematically illustrating an alternative embodiment of the present invention, in which the effecter is in the active configuration. In this Fig. the unconstrained effecter is comprised of three separate wired parts 45 or branches extruding out of the proximal end of the catheter 10.

In a preferred embodiment, at least a portion of the proximal end of the wired arms is deformed into a Zig Zag shaped configuration at least partially encircling the proximal part of the catheter 10.

As presented in FIG. 21A, the effecter may comprise more than one member e.g. three separate wired members. Each one of the effecter members, deforms into its predetermined conformation emerging from the proximal end of the bore type lumen of the catheter, and forming a three dimensional structure enclosing the main longitudinal axis of the lumen, whereby the proximal end of the effecter is directed towards the distal end of the catheter 10.

FIG. 21B illustrates a top view of the effecter as described in FIG. 21A. The top view schematically presents the upper part of the effecter being characterized by three sections 45 dispersed into three directions, encircling the outer borders of the catheter 10.

According to preferred embodiments of the invention, such an active configuration is adapted to form a defined volume within the vessel throughout which the guide wire 30 protrudes to by pass the occlusion.

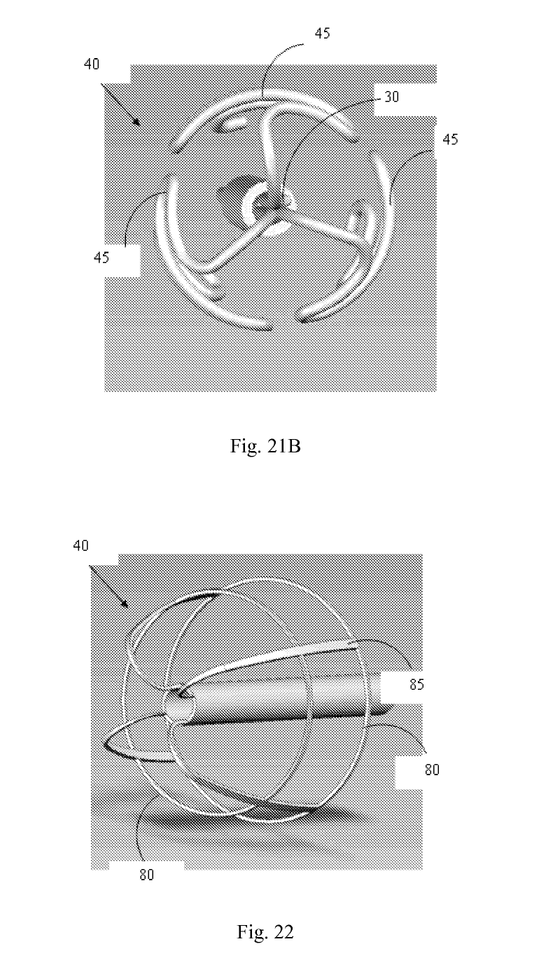

Reference is now made to FIG. 22 schematically illustrating an optional active configuration of the effecter. The effecter described in this Fig. comprises n separate loops 80 encircling the outer circumference of the catheter 10, where n is an integer greater than 1. The loops are interconnected to each other in at least one location by n wired elongated members 85 or buckled towards the distal end of the catheter 10.

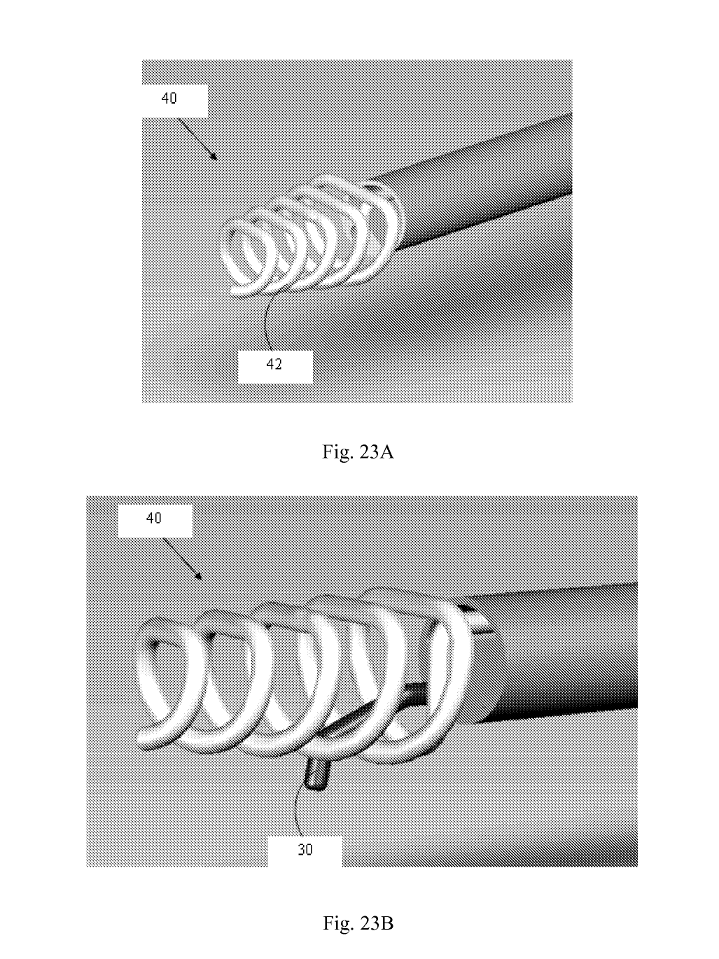

Reference is now made to FIG. 23a, schematically illustrating a catheter comprising an effecter with a spring like active configuration 40. In FIG. 23A, the spring protrudes out of the proximal end of the lumen 20, forming a spring-like shape being characterized by n helixes or loop like structures 42.

In one embodiment, the loops are characterized by a constant radius R. In another embodiment, the loops are characterized by a variable radius R which may increase, or decrease or both. As illustrated in FIG. 23B such an active configuration is adapted to define a volume throughout which the guide wire 30 may be movable.

As mentioned above, according to another embodiment of the present invention, the effecter is characterized by a diverged proximal end as illustrated in FIG. 24a.

Reference is now made to FIG. 24a schematically illustrating a catheter comprising an effecter with diverged arm-like wired elements 45. In this embodiment, the arms are diverged into a non single-point assembled manner. In this active configuration, after emergence of the effecter from the catheter confines, the guide wire 30 is adapted to be insertable throughout the space or volume defined by the armed like elements of the effecter, to enable the guide wire to penetrate the intima at said occlusion proximal end and to perform reentry at said lumen at said occlusion's distal end so as to by-pass/cross the occlusion.

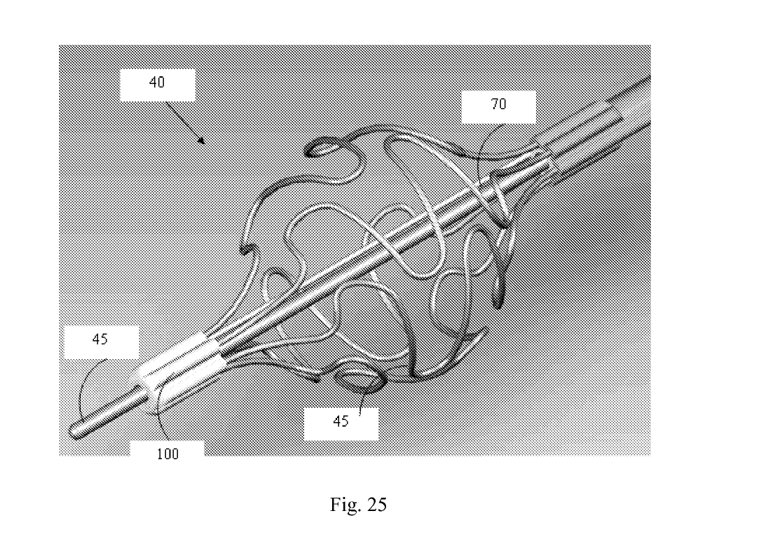

Reference is now made to FIG. 25 schematically illustrating a catheter comprising an effecter comprising several segments or parts 45, each part forming a ZIG ZAG like shaped active configuration.

It is within the scope of the invention that the wired segments define a hollow sphere like volume or space.

The wired segments protrude out of the proximal end of the catheter 20, and are re-assembled into one location 100 positioned at the proximal part of the guide wire 30, mechanically enabling the guide wire to by pass the occlusion. An actuating mechanism 70 is adapted to activate effecter. In FIG. 25 the actuating mechanism 70 is simply a wire that reciprocally move along the main longitudinal axis of the catheter such that said linear movement reconfigure the effecter 40 from an inactive configuration, in which the segments 45 are each compressed and packed together; and in the active configuration the segments 45 are deployed.

It is according to one embodiment of the invention wherein the catheter has an outer diameter of e.g., about 0.035″ to 4F (0.89-1.33 mm).

It is according to another embodiment of the invention wherein the guide-wire has a diameter between e.g., about 0.008″ to about 0.018″.

It is according to yet another embodiment of the invention wherein the effecter is at least partially made of a shape memory alloy (e.g., Nitinol) with a front segment which when unconstrained, takes on a three dimensional form to occupy space and prevent collapse of tissue into its inner space. One possible shape among many is a helix with a diameter similar to or somewhat larger than that of the suction wire or catheter. Other shapes include a variety of patterns creating a surface contour of a sphere, a cone, a cylinder or an egg-like form. The tip (front end) of the thin preformed spacing wire is a sphere with a diameter which may be larger than of the catheter, or is tightly curled up so it does not have a sharp pointing tip, but rather an atraumatic one.

It is according to yet another embodiment of the invention to provide the catheter as defined above, wherein the effecter is characterized by having a tapered shape, adapted to facilitate advancing, penetrating, anchoring, piercing, crossing or otherwise actuating of the catheter within the occlusion.

It is according to yet another embodiment of the invention to provide the catheter as defined above, wherein the guide-wire is characterized by a sharpened tip adapted for puncturetion of the occlusion and of the layers of the vessel.

It is according to yet another embodiment of the invention to provide the catheter as defined above, wherein at least a portion of the proximal portion of the effecter is a curled wire is characterized by a shape selected from a group consisting of helix, sphere, cone, cylinder, egg-like, and any combination thereof.

1. A catheter for crossing occlusions in vessels of a patient; said catheter is characterized by a main longitudinal axis, said catheter comprising:
a. at least one open-bore type lumen characterized by a distal end and a proximal end, interconnected by a main longitudinal axis, substantially parallel to said main longitudinal axis of said catheter; said distal end is located out side the body of said patient;
b. at least one guide-wire, accommodated within said lumen, reciprocally maneuvered along and around said main longitudinal axis of said lumen; said guide-wire is adapted to penetrate the intima at said occlusion proximal end and to perform reentry at said lumen at said occlusion's distal end so as to by-pass/cross said occlusion;
c. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima to perform reentry;
wherein at least a portion of said proximal end of said effecter, in said active configuration, protrudes out of said proximal end of said open-bore type lumen and is adapted to at least partially encircle at least part of the outer circumference of said proximal end of said catheter.

2. The catheter according to claim 1, wherein at least a portion of said proximal end of said effecter, in said active configuration, protrudes out of said proximal end of said open-bore type lumen and is adapted to be at least partially buckled in the direction of said distal end of said lumen whilst at least partially encircling at least part of the outer circumference of said proximal end of said catheter.

3. The catheter according to claim 1, wherein said effecter is adapted to prevent collapse of tissue into its inner space.

4. The catheter according to claim 1, wherein said at least a portion of said proximal end of said effecter is about 10% of the total length of said effecter.

5. The catheter according to claim 1, wherein at least one of said effecters comprises at least two branches, such that reconfiguration of all of said branches from said inactive configuration to said active configuration results in the at least partially encircling at least part of the outer circumference of said proximal end of said catheter.

6. The catheter according to claim 1, wherein reconfiguration from said active configuration to said inactive configuration is operatable by means of an actuating mechanism; further wherein said actuating mechanism is adapted to expose said effecter out of said lumen.

7. The catheter according to claim 6, wherein said actuating mechanism is selected from a group selected from application of heat, application of electric current, application of magnetic field, application of mechanic means or any combination thereof.

8. The catheter according to claim 6, wherein said actuating mechanism comprises at least one pushing wire; said pushing wire is mechanically connected to said effecter; such that said pushing wire is adapted to actuate said effecter.

9. The catheter according to claim 1, wherein said proximal end of said effecter is diverged in a non single-point assembled manner.

10. The catheter according to claim 1, wherein at least a portion of said proximal end of said effecter comprises n dispersed wired branches, where n is an integer greater than 1.

11. The catheter according to claim 1, wherein the shape of at least a portion of said proximal end of said effecter is selected from a group consisting of a curled wire, a spring-like shape, spiral-like shape, three dimensional configuration, a two dimensional shape, of helix, coil, spiral, sphere, cone, cylinder, egg-like, loop-like, ball-like, ovoid-like, globular-like, globe-like, orbicular-like, circular, triangle, rectangular, oval, polygonal having n ribs, n is an integer greater than 2, tapered shape, symmetric or asymmetric shape with regards to said main longitudinal axis of said lumen; and any combination thereof.

12. The catheter according to claim 1, wherein said effecter, in said inactive configuration, is maintained within said lumen such that the same is constrained to its shape by mean of said lumen.

13. The catheter according to claim 1, wherein said guide-wire is comprises a sharpened tip adapted to puncture the occlusion and/or of the layers of the vessel.

14. The catheter according to claim 1, wherein at least a portion of said proximal end of said effecter comprises n loops substantially parallel to each other, where said n is an integer greater than 1.

15. The catheter according to claim 14, wherein at least two of said loops are interconnected.

16. The catheter according to claim 1, wherein said effecter, in said inactive configuration, is disposed outside of said lumen; further wherein said proximal end of said effecter is adapted to define, in said inactive configuration, a volume throughout which said guide-wire penetrate the intima at said occlusion proximal end and performs reentry at said lumen at said occlusion's distal end thereby crosses said occlusion.

17. The catheter according to claim 1, wherein said effecter is characterized a spring-like mechanical behavior, having either a constant or a variant pitch; said spring comprises n loops, said n is an integer greater than 1.

18. The catheter according to claim 17, wherein said loops are characterized by a either a constant radius, R or a variant radius.

19. The catheter according to claim 1, wherein said effecter comprises n wires, where n is an integer greater than 1; wherein the shape of each of wires is selected from a group consisting of zig-zaged shaped, curled shaped or any combination thereof.

20. The catheter according to claim 1, wherein said catheter further comprises a vacuum generating mechanism (VGM) adapted to stabilize and fix said effecter in said active configuration within said inner surface of said vessel, to enable said guide wire to penetrate the intima at said occlusion proximal end and to perform reentry at said lumen at said occlusion's distal end so as to by-pass/cross said occlusion; said VGM is being in a fluid communication with said proximal end of said lumen; further wherein said VGM is adapted for generating an effective measure of vacuum and introducing the vacuum towards said proximal end of said catheter, via at least a portion of said lumen; such that said effecter is stabilized and fixed within the same to enable said guide-wire to penetrate the intima at said occlusion proximal end and perform reentry at said lumen at said occlusion's distal end so as to by-pass/cross said occlusion.

21. The catheter according to claim 1, wherein at least a portion of said proximal end of said effecter in said active configuration, is anchored within said inner surface of said vessel so as to enable said guide-wire to penetrate the intima at said occlusion proximal end and perform reentry at said lumen at said occlusion's distal end so as to by-pass/cross said occlusion.

22. The catheter according to claim 1, wherein at least a portion of said effecter is made of materials selected from a group consisting of shape memory material selected from a group consisting of copper-based alloy, NiTi-based materials and mixtures thereof, electro active polymers (EAPs) selected from dielectric EAPs, ferroelectric polymers, liquid crystalline polymers, ionic EAPs and any mixture thereof, soft materials, resilient materials, rubbery materials, elastic materials, flexible materials and a combination thereof.

23. A method for crossing occlusions in vessels of a patient; comprising:
a. providing at least one catheter, said catheter is characterized by a main longitudinal axis, said catheter comprising:
i. at least one open-bore type lumen characterized by a distal end and a proximal end, interconnected by a main longitudinal axis, substantially parallel to said main longitudinal axis of said catheter; said distal end is located out side the body of said patient;
ii. at least one guide-wire, accommodated within said lumen, reciprocally maneuvered along and around said main longitudinal axis of said lumen; said guide-wire is adapted to by-pass/cross said occlusion by penetrating the intima and performing reentry; and,
iii. at least one effecter having a distal end and a proximal end; said effecter is characterized by at least one active configuration and at least one inactive configuration, said proximal end of said effecter is adapted to define, in said active configuration, a volume throughout which said guide-wire penetrates the intima and performing reentry;
b. inserting said catheter to said vessel, to a location adjacent to beyond said occlusion;
c. actuating said effecter so as to reconfigure the same into said active configuration;
d. extracting said guide wire out of said lumen;
e. penetrating the intima at said occlusion's proximal end and performing reentry into the lumen at said occlusion distal end by said guide-wire; thereby crossing said occlusion;
wherein said step of actuating said effecter results in (a) the protruding of at least a portion of said proximal end of said effecter out of said lumen; and, (b) at least partially encircling at least part of the outer circumference of said proximal end of said catheter; thereby enabling said guide-wire to penetrate the intima and performing reentry so as to cross said occlusion.

24. The method according to claim 23, wherein said step of actuating said effecter results in (a) the protruding of at least a portion of said proximal end of said effecter out of said lumen; and, (b) be at least partially buckled in the direction of said distal end of said lumen whilst at least partially encircling at least part of the outer circumference of said proximal end of said catheter; thereby enabling said guide-wire to penetrate the intima and performing reentry so as to cross said occlusion.

25. The method according to claim 23, further comprises a step of removing said catheter form said patient's vessel, and, optionally maintaining said guide-wire within said vessel.

26. The method according to claim 23 further comprises a step pushing said catheter through said reentry site or said occlusion's proximal end into the lumen at said occlusion distal end for injection and visualization before its removal.

27. The method according to claim 23, further comprises a step of performing reentry with said guide-wire while applying a suction mechanism.

28. The method according to claim 23, further comprises step of providing at least one of said effecters with at least two branches, such that reconfiguration of all of said branches from said inactive configuration to said active configuration results in the at least partially encircling at least part of the outer circumference of said proximal end of said catheter.

29. The method according to claim 23, wherein reconfiguration from said active configuration to said inactive configuration is operatable by means of an actuating mechanism; further wherein said actuating mechanism is selected from a group selected from application of heat, application of electric current, application of magnetic field, application of mechanic means or any combination thereof.

30. The method according to claim 23, wherein said actuating mechanism is adapted to expose said effecter out of said lumen; further wherein said actuating mechanism comprises at least one pushing wire; said pushing wire is mechanically connected to said effecter; such that said pushing wire is adapted to actuate said effecter.

31. The method according to claim 23, wherein said proximal end of said effecter is diverged in a non single-point assembled manner, such that at least a portion of said proximal end of said effecter comprises n dispersed wired branches, where n is an integer greater than 1.

32. The method according to claim 23, wherein said effecter, in said inactive configuration, is maintained within said lumen such that the same is constrained to its shape by mean of said lumen.

33. The method according to claim 23, wherein said catheter further comprises a vacuum generating mechanism (VGM) adapted to stabilize and fix said effecter in said active configuration within said inner surface of said vessel, to enable said guide wire to reenter said lumen distally to the occlusion; further wherein said VGM is being in a fluid communication with said proximal end of said lumen; further wherein said VGM is adapted for generating an effective measure of vacuum and introducing the vacuum towards said proximal end of said catheter, via at least a portion of said lumen, such that said effecter is stabilized and fixed within the same to enable said guide-wire to puncture the intima of said vessel by-pass/cross said occlusion and to.

34. The method according to claim 23, wherein at least a portion of said proximal end of said effecter in said active configuration, is anchored within said inner surface of said vessel so as to enable said guide-wire to penetrates the intima to perform reentry so as to by-pass/cross said occlusion and to reenter the same.

35. The method according to claim 23, wherein said effecter is adapted to prevent collapse of tissue into its inner space.
